(19) United States
(12) Patent Application Publication
(10) Pub. No.: US 2022/0001124 A1
(43) Pub. Date: Jan. 6, 2022

(54) TUBE SECUREMENT SYSTEM

(71) Applicant: 3M INNOVATIVE PROPERTIES COMPANY, Saint Paul, MN (US)

(72) Inventors: JENER DE OLIVEIRA, SAO PAULO (BR); STEVEN B. HEINECKE, NEW RICHMOND, WI (US); ADRIANA S. P. LOVON, SAO PAULO (BR); FELIPE S. R. BIZARRIA, SAO PAULO (BR)

(73) Assignee: 3M INNOVATIVE PROPERTIES COMPANY, Saint Paul, MN (US)

(21) Appl. No.: 17/478,236

(22) Filed: Sep. 17, 2021

Related U.S. Application Data
(63) Continuation of application No. 16/489,723, filed on Aug. 29, 2019.
(60) Provisional application No. 62/466,009, filed on Mar. 2, 2017.

(51) Int. Cl. A61M 16/04

(52) U.S. Cl. CPC A61M 16/0497; A61M 2209/088

(57) ABSTRACT

A tube securement system is disclosed that comprises a coupling article formed from a backing layer with first major surface and a second major surface. The coupling article comprises an anchor section, a capture section, and a longitudinal portion extending along a longitudinal direction separating the anchor section from the capture section. There is a first adhesive on the first major surface of the backing at the anchor section and a second adhesive on the first major surface of the backing at the capture section. The first major surface of the backing at the longitudinal portion is free of adhesive.

### CROSS-REFERENCE TO RELATED CASES

This application is a continuation of U.S. application Ser. No. 16/489,723, filed Aug. 29, 2019, which is a national stage filing under 35 U.S.C. 371 of PCT/US2018/020145, filed Feb. 28, 2018, which claims the benefit of U.S. Provisional Application No. 62/466,009, filed Mar. 2, 2017, the disclosures of which are incorporated by reference in their entirety herein.

### FIELD

The present disclosure generally relates to a tube securement system, and methods of using same, and particularly to systems configured to be secured to skin.

### BACKGROUND

During patient treatment (e.g., at hospitals, and particularly, in Intensive Care Units (ICUs)), the insertion of tubes can be required for different purposes, such as feeding, air supply, and/or liquid removal. The tubes inserted through the mouth are referred to as endotracheal (ET) tubes and are primarily used to maintain air supply to a patient. Such ET tubes generally need to be attached to the patient's skin in order to maintain the correct position and reduce the chance of accidental extubation.

Some existing devices for ET tube securement do not allow a medical practitioner (e.g., a nurse, etc.) to intervene or evaluate the position or level of securement without removing an adhesive tape from skin, which can cause damage to the patient's skin (such as skin tears, redness, and/or damages due to constant changing of adhesives). Other existing devices are large and bulky (e.g., configured to be attached around the patient's head) and cumbersome to use, reduce patient comfort, and/or cause pressure ulcers in, e.g., a patient's mouth.

### SUMMARY

As a result, there is a need for robust, reliable, manipulatable, repositionable endotracheal tube securement systems, which provide for a standardization of procedures. The endotracheal tube securement systems of the present disclosure can, in one or more embodiments, increase the safety of ET tube securement and patient's comfort, while reducing skin damage. Systems of the present disclosure generally allow for repositioning of the ET tube and/or the system (or a portion thereof) relative to a patient (e.g., the patient's skin, the nose, cheeks, and/or an internal structure) when needed. In general, systems of the present disclosure include one or more base articles for securing the system to a patient's face, and one or more coupling articles having one or more anchor sections configured to be repositionably coupled to the one or more base articles, while also including a capture section configured to secure the coupling article to an ET tube. The one or more anchor sections of the one or more coupling articles can be repositioned on the one or more base articles without removing the one or more base articles from the skin or even changing the one or more base articles on the skin. As a result, the comfort of the ET tube securement systems described herein may be enhanced and the risk for skin damage can be reduced.

The ET tube securement systems of the present disclosure may, in one or more embodiments, also reduce pressure ulcers caused by the ET tubes, which can be a problem on patients using ET tubes. The majority of ET tubes are secured by tapes or adhesive devices that are usually changed after 24 hours which may increase the potential risk of pressure ulcers. However, by using the ET tube securement systems of the present disclosure, the medical practitioner can evaluate a potential pressure point and take actions to prevent ulcers by changing the securement device position without causing an adhesion lesion or reducing the securement of the ET tube.

The systems of the present disclosure can be provided together as a kit, e.g., the one or more base articles and the one or more coupling articles may be attached to a common delivery sheet, which can enhance manufacturability, packaging, ease-of-use and standardization of application procedures or techniques.

As a result, the systems of the present disclosure provide one or more repositionable coupling articles that can be repositioned as desired on one or more base articles that remain stably adhered to the skin until the entire system is to be changed or removed. The one or more repositionable coupling articles secure the endotracheal tube on the patient's face in order to keep it well placed. This allows site evaluation and helps reduce the potential for skin damage and pressure ulcers.

In one or more embodiments, the one or more base articles can include a release agent (e.g., a release coating) to which an adhesive on one or more anchor sections on one or more coupling articles of the system can be adhered to ensure that the one or more anchor sections of the one or more coupling articles can be removed after being first attached to the base article and then re-attached to the base article in a different location. Alternatively, or additionally, in one or more embodiments, the one or more base articles can include a first component of a mechanical fastener to which a second mating component of the mechanical fastener on the one or more anchor sections of the one or more coupling articles can be repositionably attached such that the anchor sections can be removed from a base article and re-attached to the base article in the same or a different location.

In a first aspect, one or more embodiments of an endotracheal tube securement system as described herein includes: a base article configured to be adhered to a human face, the base article having a first major surface comprising a skin-contact adhesive and a second major surface opposite the first major surface; and a coupling article comprising a first major surface and a second major surface, wherein the coupling article comprises an anchor section and a capture section arranged along a longitudinal direction; wherein the first major surface of the coupling article occupied by the anchor section comprises means for repositionably attaching the anchor section to the second major surface of the base article; wherein the first major surface of the coupling article occupied by the capture section comprises adhesive configured to secure the capture section to an endotracheal tube; and wherein the anchor section comprises a maximum lateral width measured transverse to the longitudinal direction that is greater than a maximum lateral width of the capture section.

In a second aspect, one or more embodiments of an endotracheal tube securement system described herein includes: a first base article configured to be adhered to a human face, the first base article having a first major surface comprising a skin-contact adhesive and a second major surface opposite the first major surface; a second base article configured to be adhered to a human face, the second base article having a first major surface comprising a skin-contact adhesive and a second major surface opposite the first major surface; and a coupling article comprising a first major surface and a second major surface, wherein the coupling article comprises a first anchor section, a second anchor section, and a capture section, wherein the first anchor section and the second anchor section are separate and discrete areas that are separated from each other along a lateral direction, and wherein the capture section is separated from both the first anchor section and the second anchor section along a longitudinal direction that is transverse to the lateral direction; wherein the first major surface of the coupling article occupied by the first anchor section comprises means for repositionably attaching the anchor section to the second major surface of the first base article; wherein the first major surface of the coupling article occupied by the second anchor section comprises means for repositionably attaching the second anchor section to the second major surface of the second base article; and wherein the first major surface of the coupling article occupied by the capture section comprises adhesive configured to secure the capture section to an endotracheal tube.

In a third aspect, one or more embodiments of an endotracheal tube securement system described herein includes: a base article configured to be adhered to a human face, the base article having a first major surface comprising a skin-contact adhesive and a second major surface opposite the first major surface; and a first coupling article and a second coupling article. Each of the first and second coupling articles comprises: a first major surface and a second major surface, wherein each of the first and second coupling articles comprises an anchor section and a capture section arranged along a longitudinal direction; wherein the first major surface occupied by the anchor section of each of the first and second coupling articles comprises means for repositionably attaching the anchor section to the second major surface of the base article; and wherein the first major surface occupied by the capture section of each of the first and second coupling articles comprises adhesive configured to secure the capture section to an endotracheal tube.

In a fourth aspect, one or more embodiments of an endotracheal tube securement system described herein includes: a base article configured to be adhered to a human face, the base article having a first major surface comprising a skin-contact adhesive and a second major surface opposite the first major surface; and a coupling article comprising a first major surface and a second major surface, wherein the coupling article comprises a first anchor section, a second anchor section, and a capture section, wherein the first anchor section and the second anchor section are separate and discrete areas that are separated from each other along a lateral direction, and wherein the capture section is located between the first anchor section and the second anchor section; wherein the first major surface of the coupling article occupied by the first anchor section comprises means for repositionably attaching the anchor section to the second major surface of the base article; wherein the first major surface of the coupling article occupied by the second anchor section comprises means for repositionably attaching the second anchor section to the second major surface of the base article; and wherein the first major surface of the coupling article occupied by the capture section comprises adhesive configured to secure the capture section to an endotracheal tube.

In a fifth aspect, one or more embodiments of a method of securing an endotracheal tube as described herein includes: adhesively attaching a base article on a patient using skin-contact adhesive on a first major surface of the base article; securing a capture section of a coupling article to an endotracheal tube; securing an anchor section of the coupling article to a first location on the base article, wherein the endotracheal tube is attached to the first location on the base article through the coupling article; removing the anchor section of the coupling article from the first location on the base article after securing the anchor section of the coupling article to the first location; and re-securing the anchor section of the coupling article to a second location different from the first location on the base article after removing the anchor section of the coupling article from the first location on the base article, wherein the endotracheal tube is attached to the second location on the base article through the coupling article.

In a sixth aspect, one or more embodiments of a method of securing an endotracheal tube as described herein includes: adhesively attaching a first base article on a patient using skin-contact adhesive on a first major surface of the first base article; adhesively attaching a second base article on a patient using skin-contact adhesive on a first major surface of the second base article; securing a capture section of a coupling article to an endotracheal tube; securing a first anchor section of the coupling article to a first location on the first base article, wherein the endotracheal tube is attached to the first location on the first base article through the coupling article; securing a second anchor section of the coupling article to a first location on the second base article, wherein the endotracheal tube is attached to the first location on the second base article through the coupling article; removing the first anchor section of the coupling article from the first location on the first base article after securing the first anchor section of the coupling article to the first location on the first base article; re-securing the first anchor section of the coupling article to a second location different from the first location on the second base article after removing the first anchor section of the coupling article from the first location on the first base article, wherein the endotracheal tube is attached to the second location on the first base article through the coupling article; removing the second anchor section of the coupling article from the first location on the second base article after securing the second anchor section of the coupling article to the first location on the second base article; and re-securing the second anchor section of the coupling article to a second location different from the first location on the second base article after removing the second anchor section of the coupling article from the first location on the second base article, wherein the endotracheal tube is attached to the second location on the second base article through the coupling article.

Unless specified or limited otherwise, the terms “attached,” “connected,” and “coupled,” and variations thereof, are used broadly and encompass both direct and indirect attachments, connections, and couplings.

The terms “layer,” “sheet,” and “dressing,” or variations thereof, are used to describe an article having a thickness that is small relative to its length and width.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two different monomers.

The term “repositionable” refers to the ability of an article or surface to be, at least initially, repeatedly coupled to (e.g., adhered to) and removed from a surface or substrate without substantial loss of coupling capability (e.g., adhesion) and without damage to either surface (e.g., article or underlying substrate) being coupled together. For example, a coupling article as described herein can be repositionable on a base article if the base article and the coupling article can be removed, or decoupled, from one another without causing damage to the base article or the coupling article. By way of example, some pressure-sensitive adhesives, non-tacky/cohesive polymeric materials, and mechanical fasteners may be repositionable.

The phrases “mechanical fastener” and “mechanical fastener system” generally refer to fasteners that include two mating, or engagement, surfaces or components configured to be applied to one another, each mating surface having a plurality of engagement structures or features, such that engagement structures on one mating surface are configured to engage with the engagement structures on the opposing mating surface. In some embodiments, the mechanical fastener can include two flexible mating components in the form of strips or layers. In some embodiments, the mechanical fastener can include a first mating surface or component comprising tiny, stiff protrusions shaped like hooks that are configured to engage a second mating surface or component comprising pliable loops (i.e., a “hook and loop fastener,” or “hook and pile fastener”). In some embodiments, the mechanical fastener can include inter-engaging hooks or stems (e.g., self-engaging hooks or stems) on both mating surfaces or components (i.e., a hook and hook fastener, a stem and stem fastener, or a self-engaging hook/stem fastener).

“Peel force” refers to the force needed to “peel” one surface from another surface at an angle with respect to the plane between the surfaces. Adhesive peel force can be measured using the ASTM method referenced in the “ADHESIVES” section below. Peel force between mating surfaces of a mechanical fastener can be measured using ASTM D5170-98 (2015)—Standard Test Method for Peel Strength (“T” Method) of Hook and Loop Touch Fasteners.

“Shear strength” (or “shear force”) refers to the resistance to forces that cause, or tend to cause, two contiguous parts of a body to slide relatively to each other in a direction parallel to their plane of contact. That is, shear strength is the amount of force required to move one surface relative to another surface when the two surfaces are pulled in opposite directions parallel to their plane of contact. Adhesive shear force can be measured using the ASTM method referenced in the “Adhesives” section below. Shear force between mating surfaces of a mechanical fastener can be measured using ASTM D5169-98(2015)—Standard Test Method for Shear Strength (Dynamic Method) of Hook and Loop Touch Fasteners.

As used herein, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements.

Further, the term “comprises” and variations thereof do not have a limiting meaning where these terms appear in the accompanying description. Moreover, “a,” “an,” “the,” “at least one,” and “one or more” are used interchangeably herein.

Where used herein, the transitional phrases “consists of and “consisting of exclude any element, step, or component not specified. For example, “consists of or “consisting of used in a claim would limit the claim to the components, materials or steps specifically recited in the claim except for impurities ordinarily associated therewith (i.e., impurities within a given component). When the phrase “consists of or “consisting of appears in a clause of the body of a claim, rather than immediately following the preamble, the phrase “consists of or “consisting of limits only the elements (or components or steps) set forth in that clause; other elements (or components) are not excluded from the claim as a whole.

Where used herein, the transitional phrases “consists essentially of and “consisting essentially of are used to define systems, kits and methods that include materials, steps, features, components, or elements in addition to those literally disclosed, provided that these additional materials, steps, features, components, or elements do not materially affect the basic and novel characteristic(s) of the claimed invention. The term “consisting essentially of occupies a middle ground between “comprising” and “consisting of. Further, it should be understood that the herein-described systems, kits and methods may comprise, consist essentially of, or consist of any of the herein-described components and features, as shown in the figures with or without any additional feature(s) not shown in the figures. In other words, in some embodiments, the systems, kits and methods of the present invention may have any additional feature that is not specifically shown in the figures. In some embodiments, the systems, kits and methods of the present invention do not have any additional features other than those (i.e., some or all) shown in the figures, and such additional features, not shown in the figures, are specifically excluded from the systems, kits and methods.

The above summary is not intended to describe each embodiment or every implementation of the catheter securement systems, kits, or methods of using the same as described herein. Rather, a more complete understanding of the invention will become apparent and appreciated by reference to the following Description of Illustrative Embodiments and claims in view of the accompanying figures of the drawing. Other features and aspects of the present disclosure will become apparent by consideration of the detailed description and accompanying drawings.

### BRIEF DESCRIPTION OF THE DRAWING

FIG. 1 is a plan view of one illustrative embodiment of an endotracheal tube securement system as described herein, the system shown in the form of a kit in which a coupling article and base articles are attached to a common delivery sheet.

FIG. 2 is a plan view of the first major surface of the coupling article of FIG. 1, where the first major surface is the surface facing the common delivery sheet in FIG. 1.

FIG. 3A is a cross-sectional view of the coupling article of FIG. 2 taken along line 3A-3A in FIG. 2.

FIG. 3B is a cross-sectional view of the coupling article of FIG. 2 taken along line 3B-3B in FIG. 2.

FIG. 3C is a cross-sectional view of the coupling article of FIG. 2 taken along line 3C-3C in FIG. 2.

FIG. 4 is a cross-sectional view of a base article of FIG. 1 taken along line 4-4 in FIG. 1.

FIG. 5 is a cross-sectional view of one alternative embodiment of a base article including a mechanical fastener component as described herein.

FIG. 6 is a cross-sectional view of one alternative embodiment of an anchor section of a coupling article including a mechanical fastener component as described herein.

FIG. 7 depicts the illustrative embodiment of endotracheal tube securement system 100 in use on a patient's face to support an endotracheal tube.

FIG. 8 depicts the endotracheal tube securement system 100 after removal and repositioning of the coupling article to move the endotracheal tube.

FIGS. 9-12 depict alternative illustrative embodiments of coupling articles that may be used in one or more embodiments of endotracheal tube securement systems as described herein.

FIG. 13 depicts the illustrative embodiment of coupling article 520 as depicted in FIG. 12 in use to secure an endotracheal tube.

FIG. 14 depicts another alternative illustrative embodiment of an endotracheal tube securement system as described herein.

FIG. 15A depicts the endotracheal tube securement system of FIG. 14 in use to secure an endotracheal tube.

FIG. 15B depicts the endotracheal tube securement system of FIG. 14 in use to secure an endotracheal tube having a pair of fittings protruding from a central section of the endotracheal tube.

FIG. 16 depicts one alternative illustrative embodiment of a base article that may be used in one or more embodiments of an endotracheal tube securement system as described herein.

FIG. 17 depicts one illustrative embodiment of an endotracheal tube securement system including a base article as depicted in FIG. 16 along with another alternative illustrative embodiment of a coupling article.

FIG. 18 depicts a first major surface of the coupling article depicted in FIG. 17, where the first major surface of the coupling article is that surface facing the common delivery sheet of the endotracheal tube securement system depicted in FIG. 17.

FIG. 19 depicts another alternative illustrative embodiment of an endotracheal tube securement system attached to a patient's face.

FIG. 20 depicts a first major surface of the coupling article depicted in FIG. 19, where the first major surface of the coupling article is that surface facing the patient's face.

FIGS. 21-22 depict other alternative illustrative embodiments of base articles that may be used in one or more embodiments of an endotracheal tube securement system as described herein.

FIGS. 23-25 depict other alternative illustrative embodiments of coupling articles that may be used in one or more embodiments of an endotracheal tube securement system as described herein.

FIG. 26 depicts another alternative illustrative embodiment of an endotracheal tube securement system as described herein.

FIG. 27 depicts a first major surface of the coupling article of the endotracheal tube securement system depicted in FIG. 26, where the first major surface of the coupling article is the surface facing the common delivery sheet to which the base articles and the coupling article are attached.

FIG. 28A depicts the endotracheal tube securement system depicted in FIG. 26 after attachment of the base articles and the coupling article to the patient's face.

FIG. 28B depicts the endotracheal tube securement system as attached to a patient's face as depicted in FIG. 28A after securing an endotracheal tube in position using the coupling article of the endotracheal tube securement system.

FIG. 29 depicts another alternative illustrative embodiment of a coupling article that may be used in one or more embodiments of an endotracheal tube securement system described herein.

FIG. 30 depicts the coupling article of FIG. 29 in use to secure an endotracheal tube on a patient's face.

FIG. 31 depicts another alternative illustrative embodiment of a coupling article that may be used in one or more embodiments of an endotracheal tube securement system described herein.

FIG. 32 depicts the coupling article of FIG. 31 in use to secure an endotracheal tube on a patient's face.

FIG. 33 depicts a first major surface of one illustrative embodiment of a coupling article that may be used in one or more embodiments of an endotracheal tube securement system as described herein.

FIG. 34 depicts the coupling article of FIG. 33 after attachment to a patient's face such that the coupling article is positioned to secure both a nasogastric tube and an endotracheal tube.

FIG. 35 depicts a plurality of endotracheal tube securement systems on connected common delivery sheets arranged in a roll-form.

### DESCRIPTION OF ILLUSTRATIVE EMBODIMENTS

The present disclosure generally relates to endotracheal tube securement systems and methods of using same. Particularly, endotracheal tube securement systems of the present disclosure include at least two parts or components: (i) one or more base articles that can be coupled (i.e., adhered) to skin, and (ii) one or more coupling articles having a capture section configured to secure the endotracheal tube and one or more anchor sections configured to be repositionably coupled to the one or more base articles to allow the endotracheal tube to be repositioned as desired without disrupting adhesion of the one or more base articles to skin, or requiring any portion of the one or more base articles to be removed. The one or more base articles can remain in position on the skin until it becomes necessary to change it or until the endotracheal tube is removed from the patient.

FIG. 1 depicts one illustrative embodiment of an endotracheal tube securement system 100 as described herein. By way of example only, the system 100 is shown as a kit including three elements all provided on a common delivery sheet 101. The endotracheal tube securement system 100 depicted in FIG. 1 includes a pair of base articles 110 and a coupling article 120, all of which are depicted as attached to the common delivery sheet 101.

In one or more embodiments, the common delivery sheet 101 may be in the form of a release liner having at least one surface to which adhesive articles may be releasably secured (additional details regarding potential release liners are described in greater detail below under the section entitled “RELEASE LINERS”). In one or more embodiments, the entire surface to which the adhesive articles are secured may function as a delivery sheet, while in one or more alternative embodiments, the adhesive articles may be secured to subsections of the delivery sheet 101 which are provided with release materials to allow for releasable attachment of the adhesive articles thereto. One or more embodiments of an endotracheal tube securement system 100 as described herein, when provided as a kit, may be located in a package for convenient delivery to a user. In one or more embodiments, the various components of the endotracheal tube securement system 100 in a package may be sterilized.

Although the system 100 as depicted in FIG. 1 includes two base articles 110 and one coupling article 120, other alternative embodiments of securement systems as described herein may include only one base article 110 or three or more base articles 110. Further, one or more embodiments of the systems as described herein may include two or more coupling articles 120. In one or more embodiments of securement systems as described herein, the one or more base articles 110 and/or the one or more coupling articles 120 may be provided as separate and discrete articles located in a package such that the one or more base articles 110 and/or one or more coupling articles 120 are not located on a common delivery sheet 101.

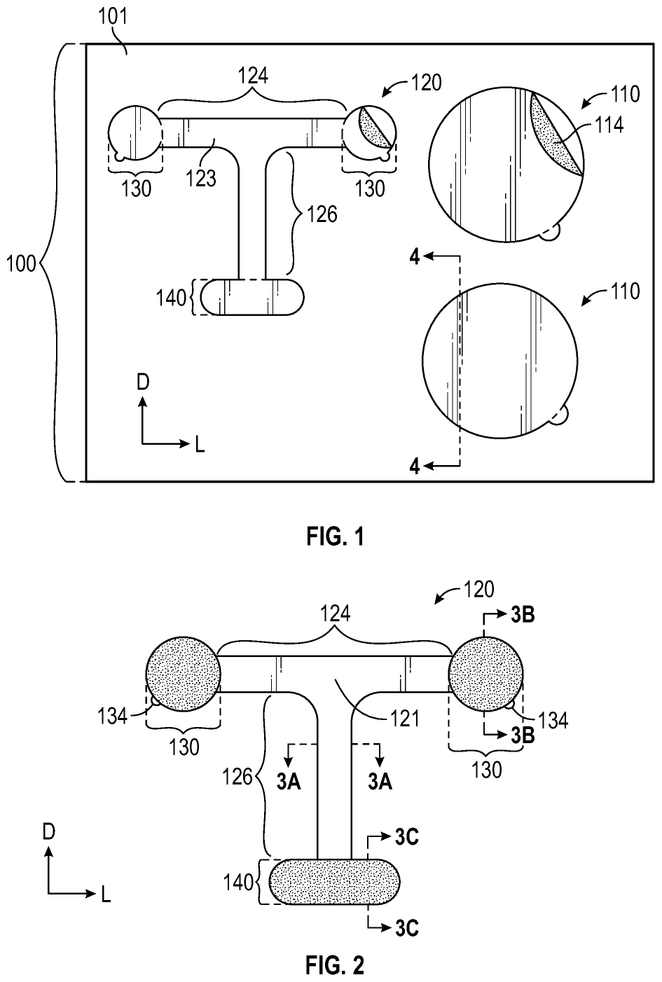

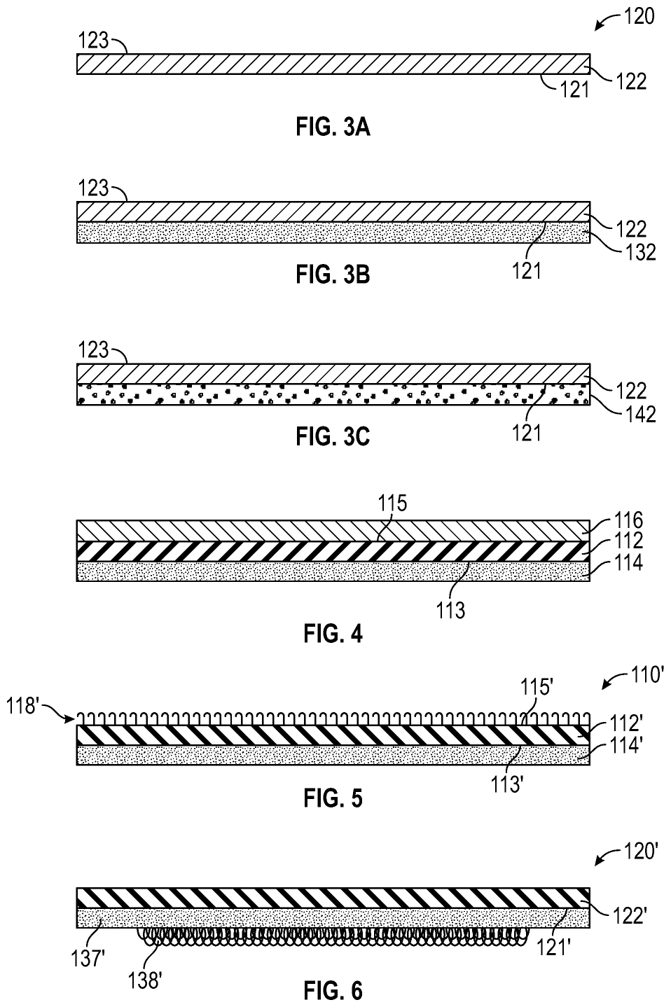

Illustrative embodiment of coupling article 120 depicted in connection with illustrative embodiment of endotracheal tube securement system 100 is depicted in connection with FIGS. 2 and 3A-3C. In particular, the first major surface 121 of the coupling article 120 is depicted in FIG. 2, while the second major surface 123 of the coupling article 120 is visible in both FIGS. 1 and 3. The depicted illustrative embodiment of coupling article 120 includes first and second anchor sections 130 and a capture section 140. The depicted illustrative embodiment of coupling article 120 includes an intermediate portion 124 extending between the first and second anchor sections 130 and a longitudinal portion 126 extending from the intermediate portion 124 to the capture section 140, with the intermediate portion 124 and the longitudinal portion 126 forming a generally T-shaped article.

Each of the anchor sections may, in one or more embodiments, include a tab 134 that may be useful in removing the anchor section 130 from the common delivery sheet 101 and/or a base article 110 to which the anchor section 130 may be attached to as described herein.

The coupling article 120 includes a first major surface facing the common delivery sheet 101 and a second major surface facing away from the common delivery sheet 101. In the depicted illustrative embodiment, the anchor sections 130 of the coupling article 120 may be described as being arranged along a longitudinal direction defined by arrow D.

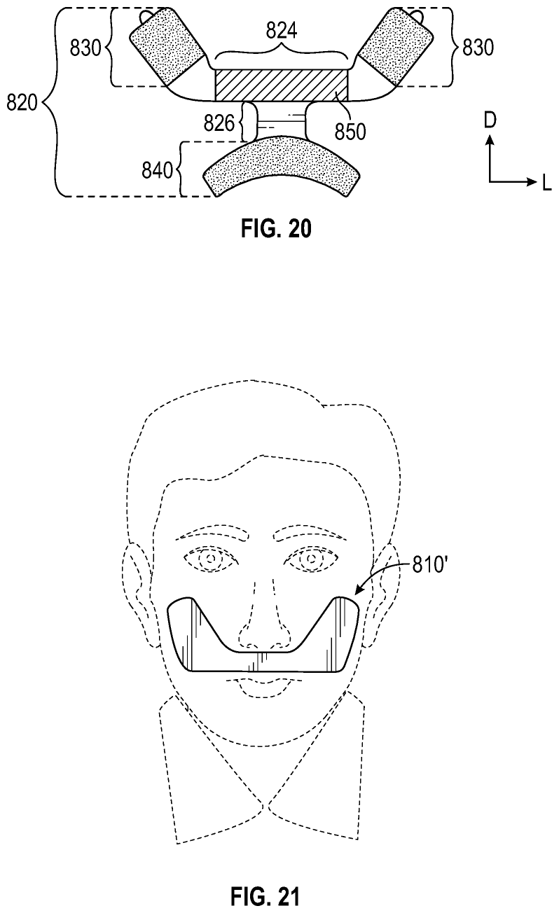

In one or more embodiments of coupling articles as described herein such as coupling article 120, the anchor section of a coupling article 120 may be described as including the intermediate portion 124 extending between the separate and discrete anchor portions 130. In such an embodiment, the anchor sections 130 could alternatively be described as separate and discrete attachment areas. In one or more embodiments, the first major surface of the intermediate portion 124 of such an anchor section may carry a hydrocolloid or other skin-friendly material to reduce the likelihood of skin irritation (e.g., as discussed in connection with the illustrative embodiments of coupling articles depicted in FIGS. 20 and 33). As a result, the anchor section of the coupling article 120 may, in one or more embodiments, be described as having a maximum lateral width measured transverse to the longitudinal direction, i.e., along the direction defined by arrow L, that is greater than a maximum lateral width of the capture section 140 where the maximum lateral width of the capture section 140 is also measured transverse to the longitudinal direction.

Alternatively, in one or more embodiments of coupling articles such as, e.g., coupling article 120 including first and second anchor sections 130, the first anchor section 130 and second anchor section 130 may be described as separate and discrete areas that are separated from each other along the lateral direction, and the capture section 140 may be described as separated from both the first anchor section 130 and the second anchor section 130 along the longitudinal direction D (i.e., transverse to the lateral direction L).

In one or more embodiments of capture articles that include first and second anchor sections 130, a geometric center of the first anchor section 130 may be described as spaced apart from a geometric center of the second anchor section 130 by a distance that is greater than a maximum lateral width of the capture section 140 as measured along the lateral direction. In one or more such embodiments, a distance from the geometric center of either of the anchor sections 130 to a geometric center of the capture section 140 may be less than the distance from the geometric center of the first anchor section 130 to the geometric center of the second anchor section 130.

Although the anchor sections 130 of coupling article 120 are shown as having the same shape, it should be understood that one or more alternative embodiments of coupling articles of securement systems as described herein may include anchor sections that have different shapes.

In the depicted illustrative embodiment of coupling article 120, the coupling article 120 includes a backing 122 as seen in, e.g., the cross-sectional view of FIG. 3A. The backing 122, although depicted as a single layer in the cross-sectional view of FIG. 3A, may be constructed of one or more layers of materials as needed to provide sufficient structural integrity to the coupling article 120. Descriptions of some potentially suitable materials for backing 122 are described below in the section entitled “Backings.”

A cross-sectional view of the illustrative embodiment of an anchor section 130 is depicted in FIG. 3B and includes, in the depicted embodiment, backing 122 along with one example of a means for repositionably attaching the anchor section 130 to a backing article that is in the form of adhesive 132 that may be used to attach the anchor section 130 to a base article 110 as described herein. In the depicted illustrative embodiment, the adhesive 132 is located on the first major surface 121 of the coupling article 120. Potentially suitable adhesives that may be used as adhesive 132 may be described below in the section entitled “Adhesives.”

A cross-sectional view of the illustrative embodiment of capture section 140 of coupling article 120 is depicted in FIG. 3C and includes, in the depicted embodiment, backing 122 along with a layer of adhesive 142 that may be used to attach the capture section 140 to an endotracheal tube as described herein. In the depicted illustrative embodiment, the adhesive 142 is located on the first major surface 121 of the coupling article 120. Potentially suitable adhesives that may be used as adhesive 142 may be described below in the section entitled “ADHESIVES.”

Returning to FIG. 1, illustrative embodiments of base articles 110 included in the depicted illustrative embodiment of endotracheal tube securement system 100 are in the form of generally circular articles, although the base articles provided in securement systems as described herein may take any number of a variety of different shapes. As discussed herein, the base articles 110 are provided with a skin contact adhesive suitable for adhering the base articles 110 to the skin of a patient. The base articles 110 can be configured (e.g., dimensioned, shaped, formed of appropriate materials, etc.) to be adhered to the skin of a patient at a selected location, e.g., on a cheek, a chin, nose, etc.

A cross-sectional view of one of the base articles 110 is depicted in FIG. 4. In the depicted illustrative embodiment, base article 110 includes a backing 112 having a first major surface 113 and a second major surface 115. The backing 112, although depicted as a single layer in the cross-sectional view of FIG. 4, may be constructed of one or more layers of materials as needed to provide sufficient structural integrity to the base article 110. Descriptions of some potentially suitable materials for a backing 112 of a base article used in a securement system as described herein are described below in the section entitled “Backings.”

A skin contact adhesive 114 is located on the first major surface 113 of the backing 112. Potentially suitable adhesives that may be used as adhesive 114 may be described below in the section entitled “Adhesives.”

Also depicted in the illustrative embodiment of base article 110 is a release layer 116 provided on second major surface 115 of backing 112. As discussed herein, release layer 116 may be used to facilitate repositioning of an adhesive provided on an anchor section of a coupling article of a securement system as described herein. In embodiments of endotracheal tube securement systems as described herein, the release layer 116 on a base article 110 may form part of a means for repositionably attaching an anchor section of a coupling article to the base article 110.

The release layer 116 may be in the form of a separate release liner attached to the backing 112 such that the release surface faces away from the backing 112. In other embodiments, the release layer 116 may take the form of, e.g., a low adhesion (low adhesion backsize, or LAB) coating provided on the backing 112 of the base article 110 in at least in a region positioned to come into contact with an anchor section of a coupling article to be used with the base article 110 in an endotracheal tube securement system as described herein. Such a low adhesion coating can allow the anchor sections of a coupling article to be repositionable on the base article as needed. One exemplary embodiment of a low adhesion backing material suitable for use with medical dressings of the present disclosure can be found in U.S. Pat. No. 5,531,855 (Heinecke et al.) and U.S. Pat. No. 6,264,976 (Heinecke et al.).

In some embodiments, the backing 112 of base articles used in endotracheal tube securement systems described herein can be formed of a stretchable material (e.g., a stretchable nonwoven, woven, film, or combination thereof) that can provide gentle removal to minimize skin damage when the system (and, particularly, the base article of the system) is removed. For example, in one or more embodiments, the base articles can include a stretch release backing (i.e., a backing formed of a stretch release material) and skin-contact adhesive, such that while stretching, there is a distribution of tension force between the backing, the skin-contact adhesive, and the skin, providing adhesive failures and reducing the tension applied on the skin as the base article is removed. Such a stretch release removal procedure may, in or more embodiments, apply a stretch removal force in a direction generally parallel to the skin surface, rather than removing the base article with a 90 degree or 180 degree peel removal process.

In some embodiments, it can be advantageous for the base article to be formed of a relatively stretchy (e.g., elastic, viscoelastic, etc.) and conformable material, while the coupling article of one or more embodiments of an endotracheal tube securement system described herein is formed of a relatively non-stretchy (e.g., inelastic, rigid, etc.) material. Such relative material properties can enhance patient comfort and/or facilitate removal of the base articles from the skin, while also ensuring enough tensile strength in the coupling articles to securely hold an endotracheal tube in a desired position.

For example, in one or more embodiments of endotracheal tube securement systems as described herein, the base articles can have a percent elongation at break (or maximum elongation) of at least 200%; in some embodiments, at least 250%; in some embodiments, at least 300%; in some embodiments, at least 400%; and in some embodiments, at least 500%.

In some embodiments, the coupling articles of one or more embodiments of endotracheal tube securement systems described herein can have a percent elongation at break of no greater than 100%; in some embodiments, no greater than 80%; in some embodiments, no greater than 75%; and in some embodiments, no greater than 50%.

Percent elongation at break can be measured using any standard tensile testing equipment known to those of ordinary skill in the art. One example of tensile testing is described in the “TEST METHODS” section below.

In one or more embodiments, the means for repositionably attaching the anchor section of a coupling article to the second major surface of a base article may include a first mechanical fastener component on the coupling article and a second mechanical fastener component on the second major surface of the base article, with the first and second mechanical fastener components being configured to attach to each other.

With reference to FIGS. 5 and 6, a cross-sectional view of one illustrative embodiment of a base article 110′ is depicted in FIG. 5, with the base article 110′ including a backing 112′ and a skin contact adhesive 114′ located on a first major surface 113′ of the backing 112′. A first component 118′ of a mechanical fastener system is depicted on the second major surface 115′ of the base article 110′. Further, in one or more alternative embodiments, a first component of a mechanical fastener system may be provided by the backing 112′ itself such that a separate component may not need to be attached to the backing 112′.

A cross-sectional view of one illustrative embodiment of an anchor section of a coupling article 120′ is depicted in cross-section in FIG. 6. The coupling article 120′ includes a backing 122′ having a second component 138′ of a mechanical fastener system attached to its first major surface 121′ by a layer of adhesive 137′. Although the second component 138′ of the mechanical fastener system is attached to the backing 122′ by adhesive 137′, it should be understood that other alternative embodiments of coupling articles including a mechanical fastener component attached thereto may have the mechanical fastener component attached by any suitable technique or combination of techniques including, e.g., welding, sewing, etc. Furthermore, in one or more embodiments, the backing 122′ may, itself, form a component of a mechanical fastener system.

Regardless of the specific construction of the mechanical fastener components, they may be provided and function to allow for repositionable attachment of an anchor section of a coupling article to a base article of an endotracheal tube securement system as described herein.

The means for repositionably attaching the anchor section of a coupling article to the second major surface of a base article in endotracheal tube securement systems as described herein may alternatively be in the form of non-tacky/cohesive polymeric materials that allow for repositioning of articles after an initial attachment.

For example, a layer of non-tacky/cohesive polymeric material may be provided on the anchor sections of coupling articles in the place of the adhesive 132 described in connection with the coupling article 120 depicted in the cross-sectional view of FIG. 3B. Backing layer 112 of the base article 110 may, in one or more embodiments, present a surface to which the non-tacky/cohesive polymeric material on the coupling article 120 attaches without the addition of a selected attachment layer or other treatment. In one or more alternative embodiments, the backing layer 112 of the base article 110 may include target materials (e.g., a layer, film, etc.) to which the non-tacky/cohesive polymeric material on the coupling article 120 attaches. In still one or more other alternative embodiments, a layer of non-tacky/cohesive polymeric material may be provided on the second major surface 115 of the backing layer 112 of the base article 110 (in place of or in addition to, e.g., release layer 116) while the first major surface 121 of the backing 122 of the coupling article 120 includes materials to which the non-tacky/cohesive polymeric material on the base article 110 attaches.

The non-tacky/cohesive polymeric materials when used on the coupling articles and/or base articles of endotracheal tube securement systems as described herein provide for repositionable attachment of the coupling articles on base articles as described herein. Examples of some potentially suitable non-tacky/cohesive polymeric materials or attachment systems that may provide for repositionable attachment of the coupling articles on the base articles as described herein may be described in, e.g., EP 0443263 (Miller et al.) and/or U.S. Pat. No. 5,888,335 (Kobe et al.); U.S. Pat. No. 5,908,695 (Kobe et al.); U.S. Pat. No. 6,004,670 (Kobe et al.); and U.S. Pat. No. 7,135,213 (Maki et al.).

Furthermore, whether the means for repositionable attachment of an anchor section of a coupling article of an endotracheal tube securement system as described herein takes the form of an adhesive, non-tacky/cohesive polymeric material, or a mechanical fastener system, the attachment is preferably sufficiently strong in order to provide reliable securement of an endotracheal tube for the desired period of time. That is, whether an adhesive, non-tacky/cohesive polymeric material, mechanical fastener system, or another coupling means is employed between the base article and coupling article, the attachment between the coupling article and the base article is preferably configured such that the force required to remove (e.g. peel, etc.) the coupling article from the base article is relatively low to allow easy repositioning of the coupling article to the base article as necessary, while the shear strength between the layers is relatively high to ensure adequate securement of an endotracheal tube. Further, removal of the coupling article from the base article preferably does not result in removal of the base article from the skin of a patient unless removal of the base article along with the coupling article is specifically desired by a user.

In addition, although the means for repositionable attachment of an anchor section of a coupling article to a base article in an endotracheal tube may take only one form, e.g., only adhesive, only non-tacky/cohesive polymeric materials, only a mechanical fastener system, etc., one or more alternative embodiments of the endotracheal tube securement systems described herein may include an anchor section of a coupling article that includes two or more means for repositionable attachment, e.g., two or more of an adhesive, non-tacky/cohesive polymeric material, mechanical fastener system, etc.

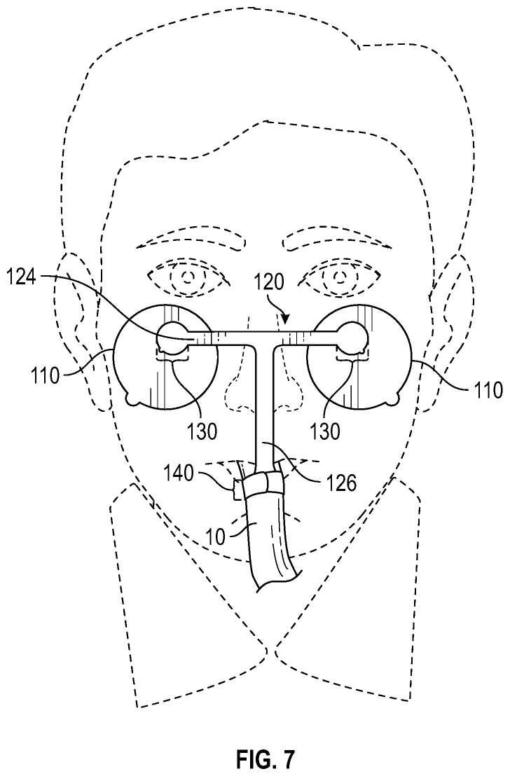

The base articles 110 and coupling article 120 of endotracheal tube securement system 100 are depicted on the face of a patient in FIG. 7, where the endotracheal tube securement system 100 is used to secure endotracheal tube 10 on the patient. The base articles 110 are, in the depicted arrangement, located on the cheeks of the patient while the anchor sections 130 of coupling article 110 are attached to the base articles 110 such that an intermediate portion 124 of coupling article 120 that extends between anchor sections 130 spans the patient's nose. Further, the longitudinal portion 126 of the coupling article 120 that extends from the intermediate portion 124 to the capture section 140 extends downward such that capture section 140 can be attached to the endotracheal tube 10 extending out of the patient's mouth.

As shown in the illustrative embodiment of the endotracheal tube securement system depicted in FIGS. 1 and 7, one or more embodiments of the anchor sections 130 of coupling article 120 can have a shape that mimics the shape of the base articles 110, while also generally being smaller than the base article 110. Coupling articles 120 having anchor sections 130 that mimic the shape of the base articles 110 (e.g., being generally circular in the case of illustrative embodiments of base articles 110 and coupling articles 120) and that can be contained within the areas of the base article 110 when the coupling article 120 is attached to the base articles 110 can reduce the chances of attaching the coupling article 120 directly to the patient's skin outside of the boundaries of the base articles 110.

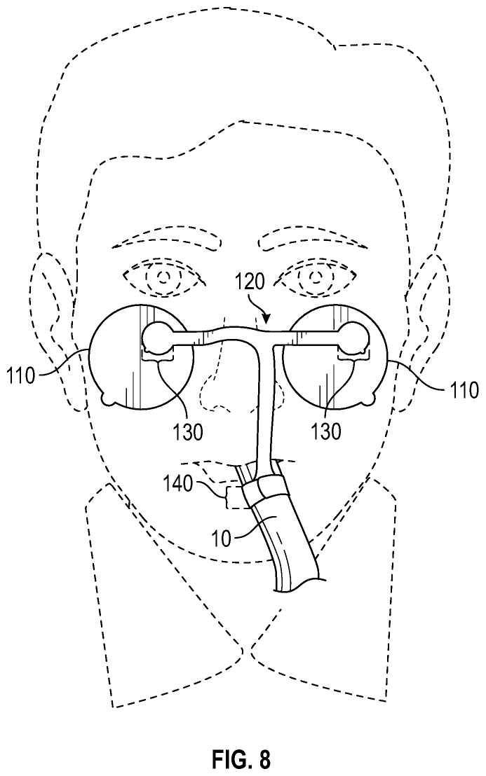

Furthermore, having smaller anchor sections 130 may allow for repositioning of the coupling article 120 with respect to the base articles 110 to facilitate shifting or changing the position of the endotracheal tube 10 without requiring movement of the base articles 110. One example of this feature is depicted in FIG. 8 where the endotracheal tube 10 has been shifted to the left on the patient's face. Correspondingly, the anchor sections 130 of coupling article 120 are shifted to the left on their respective base articles 110, while the base articles 110 remain in the same position on the cheeks of the patient.

In one or more embodiments, the base articles of endotracheal tube securement systems as described herein may be described as having a base article footprint area while the anchor section of the coupling article may be described as having an anchor section footprint area. In one or more embodiments, the anchor section footprint area is smaller than the base article footprint area such that placement of an anchor section on the base article may be facilitated. In one or more embodiments, the anchor section footprint area of the coupling articles may be 90% or less of the base article footprint area. In one or more alternative embodiments, the anchor section footprint area of the coupling articles may be 80% or less, 70% or less, or 60% or less of the base article footprint area. At the lower end of the range, it may be preferred that the anchor section may be large enough to occupy 10% or more, 20% or more, or 30% or more of the base article footprint area.

Regardless of the relative sizes of the base article footprint area and the anchor section footprint area of securement systems as described herein, the size of the anchor section footprint areas of coupling articles described herein are preferably large enough to resist unwanted detachment of the coupling articles from base articles to which they are attached to secure an endotracheal tube on a patient.

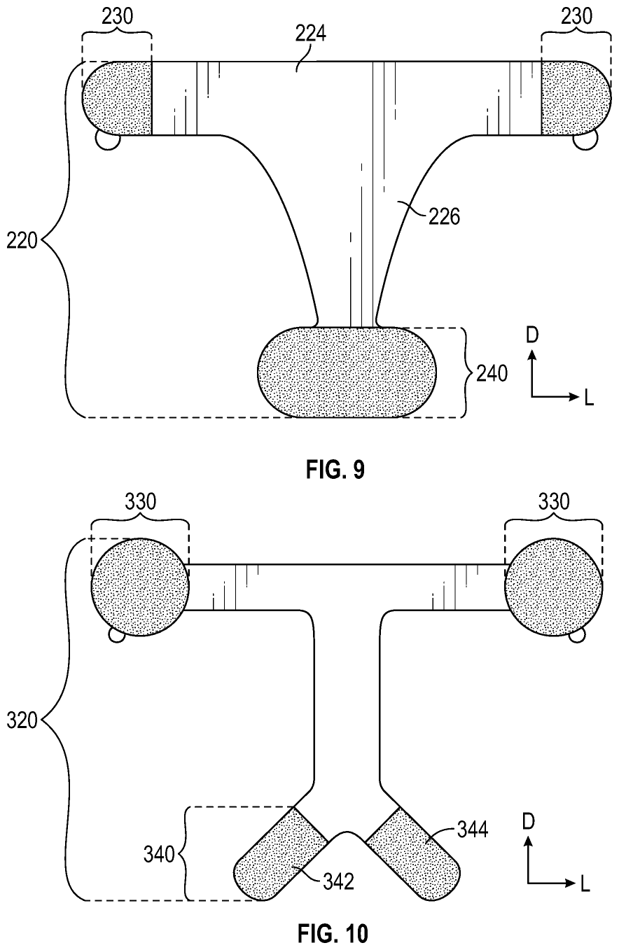

FIG. 9 depicts a first major surface of another illustrative embodiment of a coupling article 220 that may be used in one or more embodiments of an endotracheal tube securement system as described herein. This illustrative embodiment of coupling article 220 is provided to show that the shape of the coupling article 220 can vary. The coupling article 220 includes first and second anchor sections 230, along with a capture section 240, with the anchor sections 230 and the capture section 240 being connected to each other by a wider longitudinal portion 226 that extends downward from the intermediate portion 224 extending between anchor sections 230. The longitudinal portion 226 tapirs as it approaches capture section 240 in this illustrative embodiment.

FIG. 10 depicts a first major surface of another illustrative embodiment of a coupling article 320 that may be used in one or more embodiments of an endotracheal tube securement system as described herein. This illustrative embodiment of coupling article 320 is provided to show that the capture section 340 may take a variety of different forms. In particular, the capture section 340 of coupling article 320 is generally Y-shaped and includes a pair of attachment areas 342 and 344 which may include adhesive used to secure the coupling article 320 to an endotracheal tube. Although the Y-shaped capture section 340 includes two separate and discrete areas of adhesive 342 and 344, one or more alternative embodiments may include a capture section 340 that includes a single contiguous area of adhesive used to secure an endotracheal tube to the coupling article 320. This depicted embodiment of coupling article 320 also includes a pair of anchor sections 330 similar to those described above in connection with coupling articles 120 and 220.

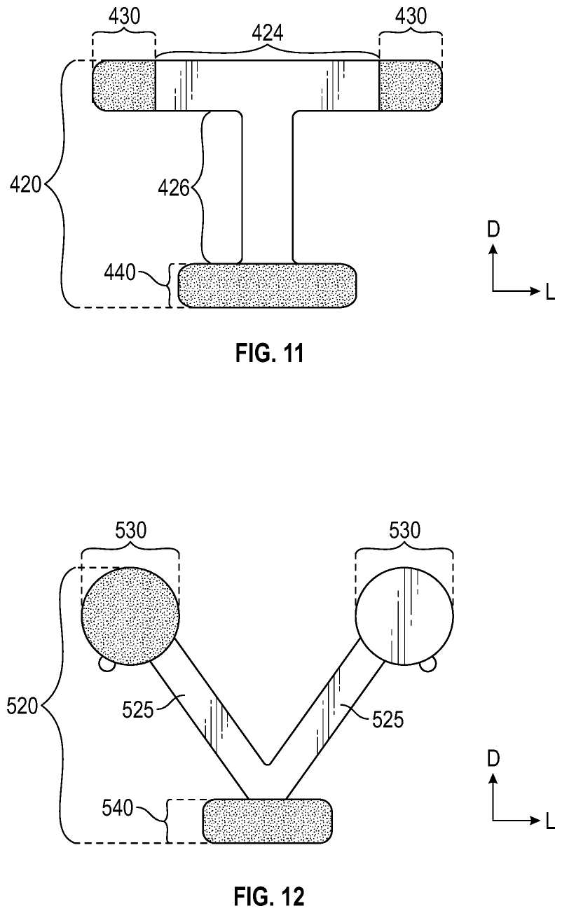

FIG. 11 depicts the first major surface of still another illustrative embodiment of a coupling article 420 including anchor sections 430 attached to each other by an intermediate portion 424 extending between the anchor sections 430, and a capture section 440 attached to the intermediate portion 424 of the coupling article 420 by a longitudinal portion 426. This illustrative embodiment of coupling article 420 depicts a more rectilinear shaped coupling article.

Another optional feature depicted in connection with coupling article 420 is a layer of a hydrocolloid that may be provided on at least a portion of the first major surface of the coupling article 420 between the anchor sections 430 and/or the capture section 440. The use of a hydrocolloid on the first major surface of the coupling article 420 may reduce the likelihood of skin irritation where the coupling article 420 contacts the skin of a patient as it extends between base articles and an endotracheal tube during use.

FIG. 12 depicts the first major surface of yet another illustrative embodiment of a coupling article 520 including anchor sections 530 and a capture section 540. One difference between this illustrative embodiment and others described herein is that each of the anchor sections 530 is connected to the capture section 540 by a leg 525 that extends directly from each of the anchor sections 530 to the capture section 540. No portion of the coupling article 520 extends directly between the anchor sections 530. One potential advantage of such a construction may be limiting contact between the coupling article 520 and the nose of a patient.

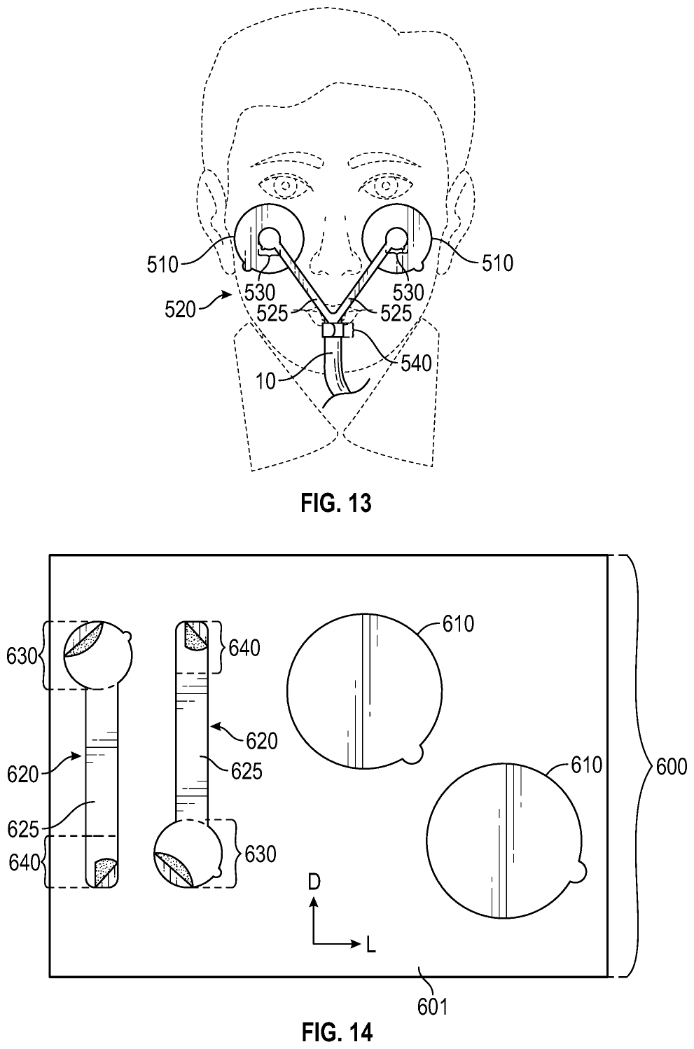

In particular, the coupling article 520 is depicted in use in FIG. 13 where anchor sections 530 are attached to base articles 510 on opposite sides of the patient's nose. Legs 525 of coupling article 520 extend downward from the base articles 510 to the endotracheal tube 10, which is attached to the coupling article 520 using capture section 540. The legs 525 extend on each side of the patient's nose, and do not cross over the patient's nose as discussed herein.

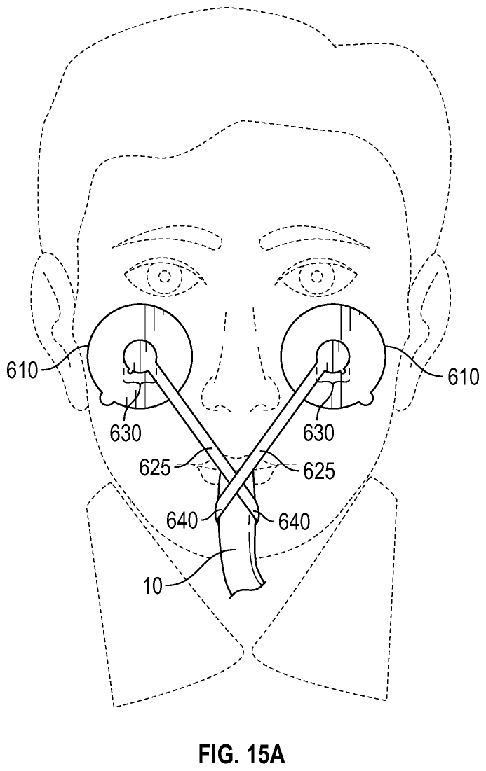

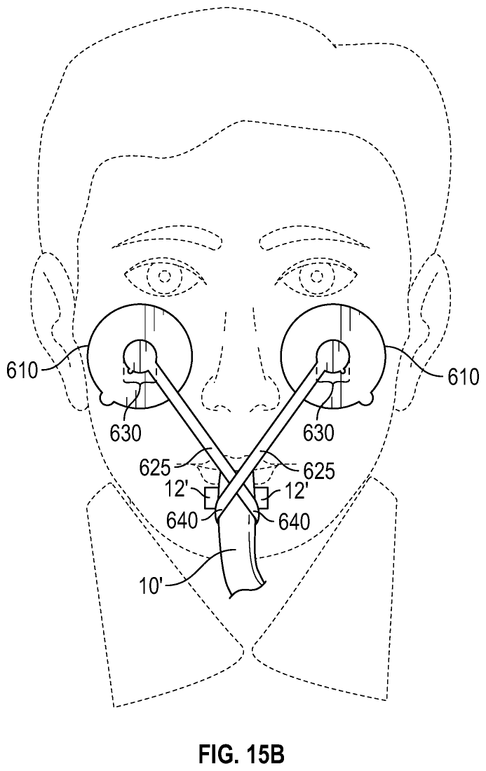

Another illustrative embodiment of an endotracheal tube securement system 600 is depicted in FIG. 14. The securement system 600 includes a common delivery sheet 601 to which a pair of base articles 610 and a pair of coupling article 620 are attached. Base articles 610 may, in many respects, be similar to base articles 110 as discussed in connection with securement system 100.

One difference between securement system 600 and securement system 100, however, is that securement system 600 includes a pair of coupling articles 620. Each of the coupling articles 620 includes an anchor section 630 and a capture section 640, with the anchor section 630 being connected to the anchor section 640 by a leg 625 extending from the anchor section 630 to the capture section 640. The shapes of the anchor sections 630 and capture section 640 may take many different forms other than those depicted in connection with the illustrative embodiments of coupling articles 620.

The securement system 600 as depicted in FIG. 14 is depicted in use in FIG. 15A, where an anchor section 630 of each of the coupling article 620 is depicted as attached to one of the base articles 610 while the capture sections of the coupling articles 640 are depicted as wrapping around and attached to an endotracheal tube 10. The endotracheal tube 10 is supported by the coupling articles 620 by legs 625 that extend from the anchor section 630 on base articles 610 to the capture sections 640 attached to the endotracheal tube 10.

The securement system 600 as depicted in FIG. 14 is also depicted in use in FIG. 15B in connection with an endotracheal tube 10′ that includes fittings 12′ that extend outwardly from the central portion of endotracheal tube 10′. When used in connection with an endotracheal tube 10′ that includes fittings 12′, the coupling articles 620 may advantageously include capture section 640 that wrap around (at least partially) the fittings 12′ where the capture sections 640 are attached to the endotracheal tube 10′. As a result, support of the endotracheal tube 10′ by the legs 625 of coupling articles 620 may at least partially be supported in tension along legs 625 and rely less on shear forces generated by adhesive on capture sections 640 where attached to endotracheal tube 10 as seen in, e.g., FIG. 15A.

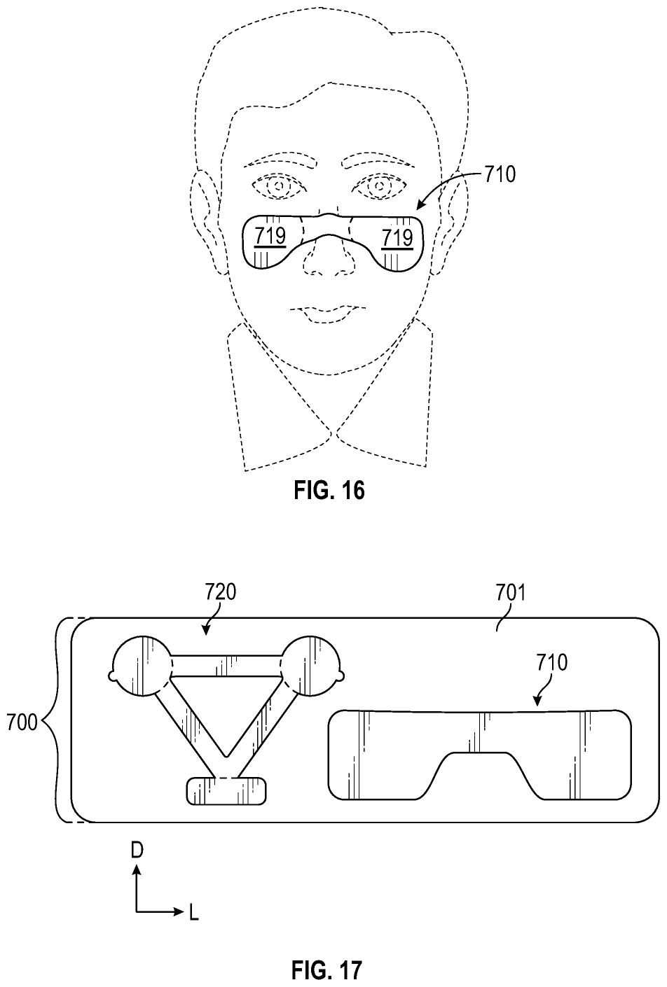

As discussed herein, the coupling articles used in endotracheal tube securement systems of the present invention may take a variety of different forms. Similarly, the base articles used in one or more embodiments of endotracheal tube securement systems as described herein may also take a variety of different forms. One illustrative embodiment of a base article 710 that may be provided in one or more embodiments of securement systems as described herein is depicted in FIG. 16.

The base article 710 depicted in FIG. 16 may include two separate areas of skin contact adhesive 719 located on, e.g., opposite sides of a patient's nose such that the intermediate portion of the base article 710 located between skin contact adhesive areas 719 is, in one or more embodiments, not attached to the skin of the patient. Alternatively, the portion of the first major surface of the base article 710 facing the patient's skin between the skin contact adhesive areas 719 may be coated with a hydrocolloid or other skin friendly material to, in one or more embodiments, potentially reduce irritation of the patient's skin.

The base article 710 depicted in FIG. 16 is depicted in connection with another alternative embodiment of an endotracheal tube securement system 700 in FIG. 17. The base article 710 is depicted along with another illustrative embodiment of a coupling article 720, with both the base article 710 and the coupling article 720 attached to a common delivery sheet 701.

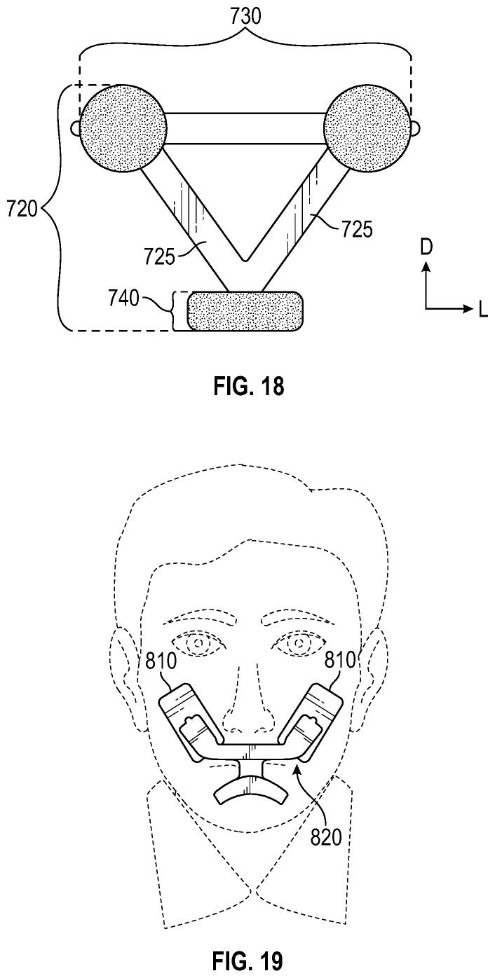

The first major surface of the coupling article 720 is attached to and faces the common delivery sheet 701 as depicted in FIG. 17. The first major surface of the coupling article 720 is, however, depicted in the view of FIG. 18, where the coupling article 720 includes an anchor section 730 attached to a capture section 740 by legs 725. One difference between the coupling article 720 and some of the coupling articles described above is that anchor section 730 spans the lateral width of the coupling article 720 along the direction L. Further, adhesive or another means for repositionably attaching the anchor section 730 to the base article 710 may occupy substantially the entire lateral width of the coupling article 720. In such an embodiment, a base article 710 that also spans the patient's nose as seen in, e.g., FIG. 16 may be useful in reducing the likelihood of skin irritation caused by the anchor section 730 of the coupling article 720.

Another illustrative embodiment of an endotracheal tube securement system as described herein is depicted in connection with FIGS. 19 and 20. The depicted securement system includes a pair of base articles 810 and a coupling article 820 configured to attached to the base articles 810 to support an endotracheal tube. In particular, the coupling article 810 is depicted as attached to base articles 810 on the face of a patient in FIG. 19, with the coupling article 820 extending between the base articles 810 below the nose of the patient.

The first major surface of the coupling article 820 is depicted in FIG. 20 where the anchor sections 830 which, in the depicted embodiment, include an adhesive, are depicted along with a capture section 840. An intermediate portion 824 of the coupling article 820 extends between the anchor sections 830, with a longitudinal portion 826 extending downward from the intermediate portion 824 to attach the capture section to the intermediate portion 824 and, thus, to the anchor sections 830.

Another optional feature of one or more embodiments of coupling articles of securement systems as described herein is depicted in FIG. 20 in the form of a hydrocolloid layer 850 being located on at least a portion of the intermediate portion 824 of the coupling article 820. Although the hydrocolloid 850 is depicted as being located on a limited portion of the intermediate portion 824 of the coupling article 820, it should be understood that the hydrocolloid 850 may extend over substantially all of the intermediate portion 824 between the anchor sections 830. Furthermore, in one or more embodiments, the hydrocolloid 850 may also be found on the longitudinal portion 826 of the coupling article 820. As discussed herein, the use of a hydrocolloid or other skin friendly material on the first major surface of the coupling articles may help to reduce skin irritation in those areas of the coupling articles that are not attached to a base article and/or an endotracheal tube.

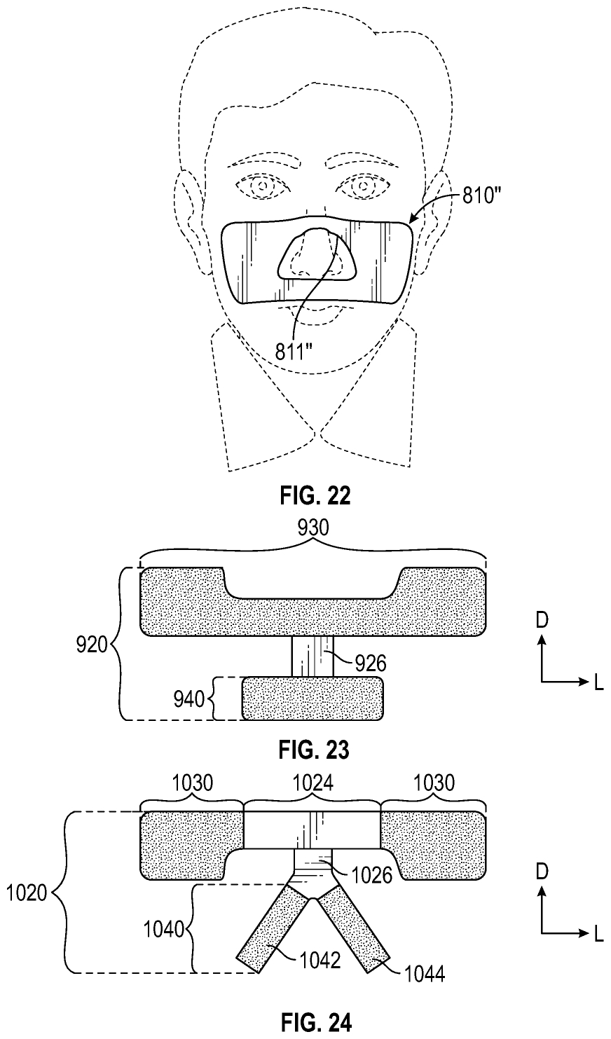

As discussed herein, one or more embodiments of endotracheal tube securement systems of the present disclosure may include base articles having a variety of shapes. Two examples of potentially useful shapes for various embodiments of base articles used in such securement systems are depicted in FIGS. 21 and 22. In particular, base article 810′ as depicted in FIG. 21 includes portions that extend upward onto a patient's cheeks with a central portion extending between the cheek portions along the upper lip of the patient below the patient's nose. The illustrative embodiment of base article 810″ as depicted in FIG. 22 includes an aperture 811″ such that the base article 810″ extends both over the bridge of the patient's nose as well as underneath the patient's nose along the upper lip with both of those intermediate portions connecting areas of the base article 810″ that are located on the cheeks of the patient.

Additional illustrative embodiments of coupling articles that may be used in one or more embodiments of endotracheal tube securement systems as described herein are depicted in connection with FIGS. 23-25. The first major surface of one illustrative embodiment of a coupling article 920 is depicted in FIG. 23. The coupling article 920 includes an anchor section 930 and a capture section 940, with the capture section 940 being attached to the anchor section 930 by an intermediate portion 926 extending between the anchor section 930 and the capture section 940. This particular illustrative embodiment of capture article 920 may be particularly useful when the anchor section 930 is located on the upper lip of a patient and could be used with, for example, the base articles 810′ and 810″ as depicted in FIGS. 21 and 22, respectively.

The first major surface of another illustrative embodiment of a coupling article 1020 is depicted in FIG. 24. The coupling article 1020 includes a pair of separated and discrete anchor sections 1030 with an intermediate portion 1024 extending between the anchor sections 1030. The coupling article 1020 further includes an intermediate portion 1026 which attaches a capture section 1042 the intermediate portion 1024 of the coupling article 1020. The depicted capture section 1040 includes a pair of attachment areas 1042 and 1044 that may be used to attach the capture section 1040 to an endotracheal tube as described herein.

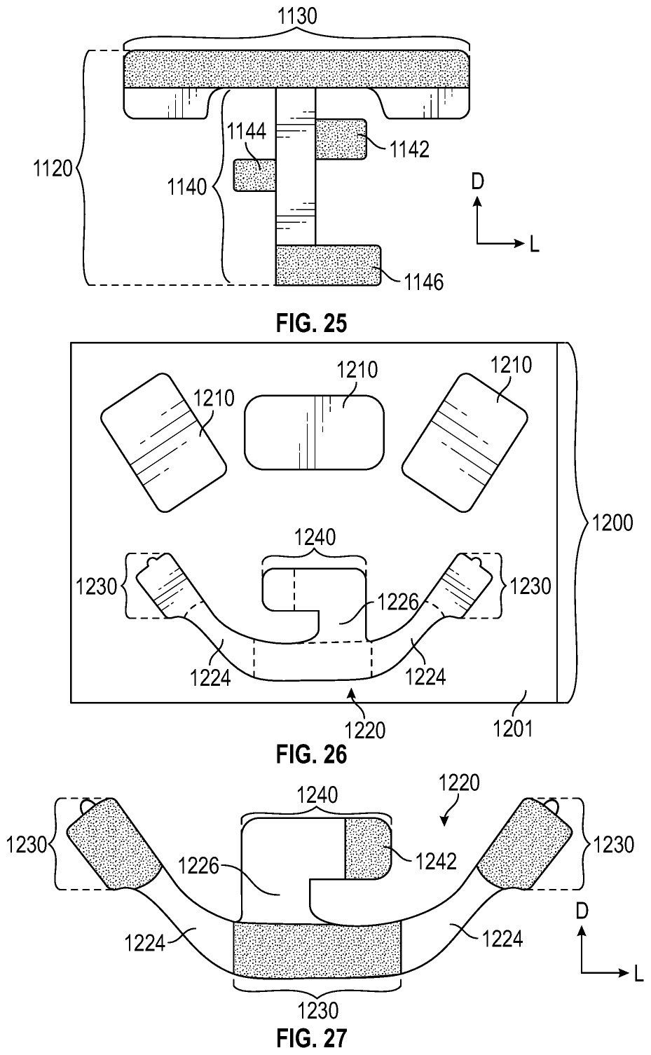

The first major surface of still another illustrative embodiment of a coupling article 1120 is depicted in FIG. 25. The coupling article 1120 includes an anchor section 1130 and a capture section 1140. In particular, the capture section 1140 includes attachment areas 1142, 1144, and 1146. The use of multiple attachment areas in the capture section 1140 may be useful in providing additional security in the attachment of capture section 1142 and endotracheal tube.

Another illustrative embodiment of an endotracheal tube securement system as described herein is depicted in connection with FIGS. 26-28B. The depicted embodiment of endotracheal tube securement system 1200 as depicted in FIG. 26 includes three base articles 1210 and a coupling article 1220, all of which are attached to a common delivery sheet 1201.

Referring to, e.g., FIG. 27 where the first major surface of the coupling article 1220 is depicted, the coupling article 1220 includes anchor sections 1230 located on opposite ends of the intermediate portion 1224 of the coupling article 1220, with an intermediate anchor section being located between the distal anchor sections 1230 located on opposite ends of the intermediate portion 1224 of the coupling article 1220.

The coupling article 1220 further includes a capture section 1240 attached to the intermediate portion 1224 of the coupling article 1220 by a longitudinal portion 1226. The capture section 1240 includes adhesive 1242 configured to attach the capture section 1240 to an endotracheal tube as discussed herein.

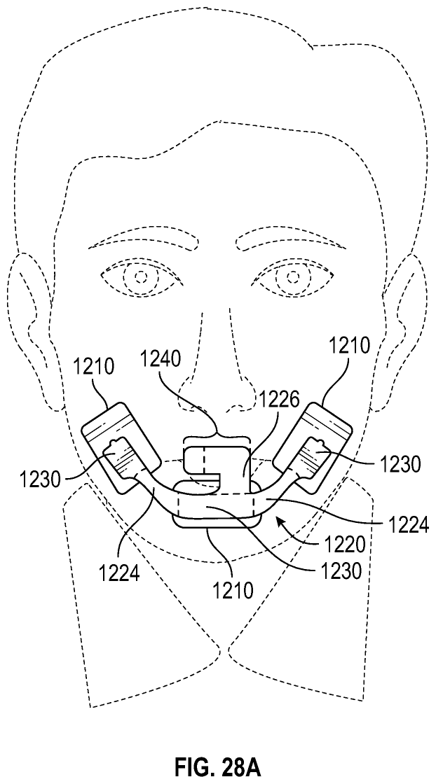

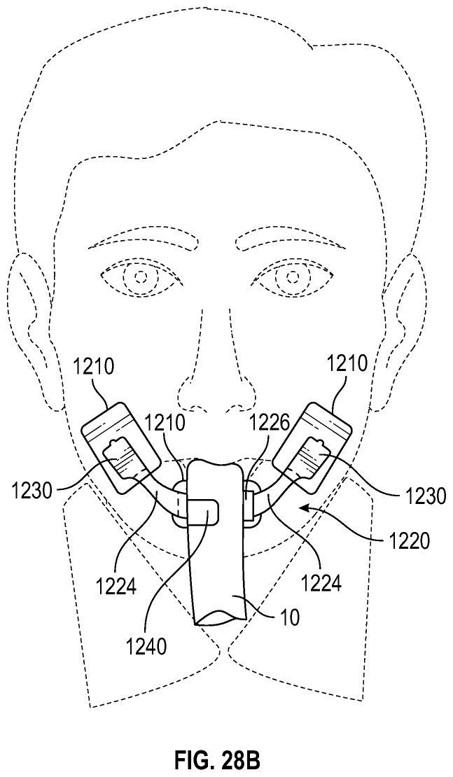

In use as depicted in, e.g., FIG. 28A, the base articles 1210 may be attached to the skin of the patient such that one of the base articles 1210 is located below the patient's mouth while the remaining pair of base articles 1210 are located on opposite cheeks of the patient. The coupling article 1220 may then be attached to the base articles 1210 with the distal anchor sections 1230 being attached to the base articles 1210 positioned on the patient's cheeks, while the intermediate anchor section 1230 is attached to the base article 1210 located on the patient's chin.

The endotracheal tube securement system 1200 is depicted in use to secure an endotracheal tube 10 in FIG. 28B with the intermediate portion 1226 of the coupling article 1220 being folded downward such that a portion of the capture section 1240 of the coupling article 1220 is located between the base article 1210 on the patient's chin and the endotracheal tube 10, with a portion of the capture section 1240 wrapping around the endotracheal tube 10 as seen in FIG. 28B.

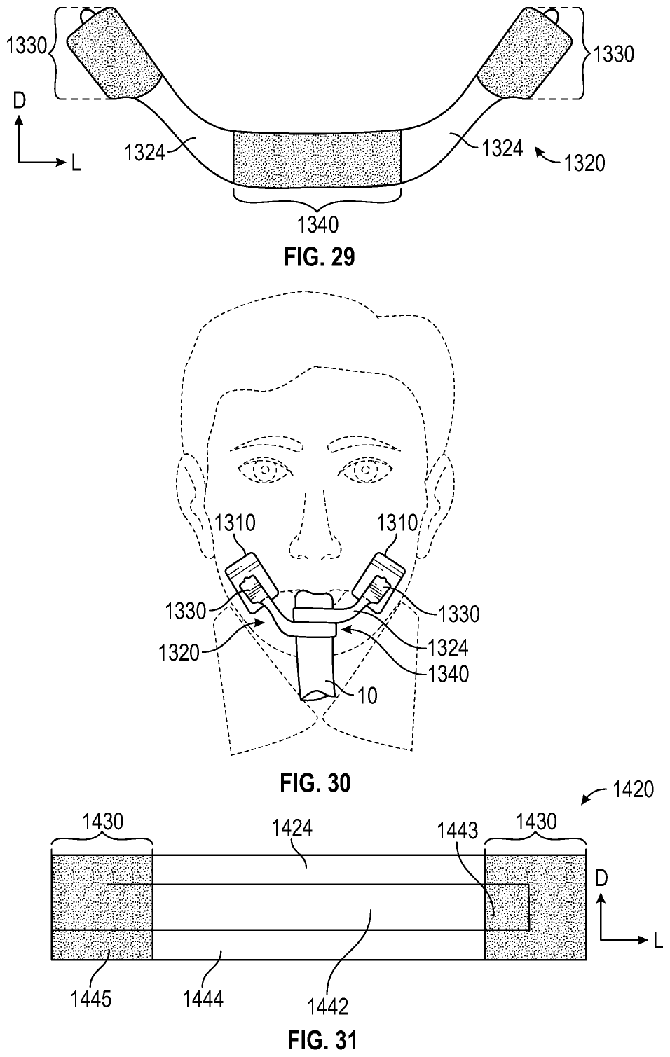

The first major surface of another illustrative embodiment of a coupling article 1320 is depicted in FIG. 29. The coupling article 1320 includes a pair of separated and discrete anchor sections 1330 with an intermediate portion 1324 extending between the anchor sections 1330. The anchor sections 1330 of the illustrative embodiment of coupling article 1320 are separate and discrete areas that are separated from each other along the lateral direction L. In the depicted illustrative embodiment of coupling article 1320, the capture section 1340 is located between the first anchor section 1330 at one end of the coupling article 1320 and the second anchor section 1330 at the opposite end of the coupling article 1320.

The depicted embodiment of coupling article 1320 is depicted in use to secure an endotracheal tube 10 on a patient's face in FIG. 30. In particular, the capture section 1340 of coupling article 1320 is wrapped around the endotracheal tube 10, while the anchor sections 1330 are attached to base articles 1310 located on the patient's face. One potential advantage of an embodiment of a coupling article 1320 having a capture section 1340 configured to wrap around an endotracheal tube 10 is that retention of the endotracheal tube 10 may be, at least in part, a function of the tensile strength of the intermediate portion 1324 and the capture section 1340 of coupling article 1320 rather than being limited to the shear forces generated by adhesive on the capture section 1340.

The first major surface of another illustrative embodiment of a coupling article 1420 is depicted in FIG. 31. The coupling article 1420 includes a pair of separated and discrete anchor sections 1430 with an intermediate portion 1424 extending between the anchor sections 1430. The anchor sections 1430 of the illustrative embodiment of coupling article 1420 are separate and discrete areas that are separated from each other along the lateral direction L. In the depicted illustrative embodiment of coupling article 1420, the capture section includes a first leg 1442 attached to a first anchor section 1430 and terminating proximate the second anchor section 1430 at the opposite end of the coupling article 1420. The capture section further includes a second leg 1444 attached to the second anchor section 1430 and terminating proximate the first anchor section 1430 at the opposite end of the coupling article 1420.

As such, the capture section of this illustrative embodiment of the coupling article 1420 may be described as including both the first leg 1442 and second leg 1444 and being located between the first anchor section 1430 at one end of the coupling article 1420 and the second anchor section 1430 at the opposite end of the coupling article 1420.

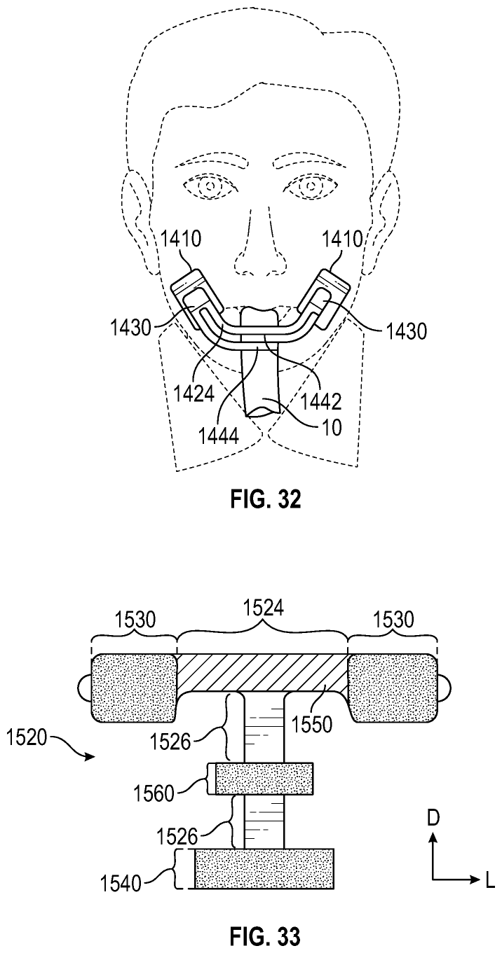

The depicted embodiment of coupling article 1420 is depicted in use to secure an endotracheal tube 10 on a patient's face in FIG. 32. In particular, the first leg 1442 of the capture section of coupling article 1420 is wrapped around the endotracheal tube 10 from one end of the coupling article 1420 and the second leg 1444 of the capture section of coupling article 1420 is wrapped around the endotracheal tube 10 from an opposite end of the coupling article 1420. The anchor sections 1430 are attached to base articles 1410 located on the patient's face.

In the depicted embodiment of coupling article 1420, the adhesive used to secure anchor sections 1430 to base articles 1410 May be the same as the adhesive used to attach the first leg 1442 and the second leg 1444 of the capture section of coupling article 1420 to the endotracheal tube 10. In one or more embodiments, a first portion of the adhesive of the capture section may be described as being located on the first leg 1442 and a second portion of the adhesive of the capture section may be described as being located on the second leg 1444.

The first major surface of yet another illustrative embodiment of a coupling article that may be used in one or more embodiments of an endotracheal tube securement system as described herein is depicted in FIG. 33. The coupling article 1520 includes a pair of anchor sections 1530 separated from each other by an intermediate portion 1524 along the lateral direction L. In one or more embodiments, the first major surface of the intermediate portion 1524 of the coupling article 1520 may include a skin friendly material 1550 such as, e.g., a hydrocolloid to reduce the likelihood of skin irritation by that portion of the coupling article 1520.

This depicted illustrative embodiment of coupling article 1520 includes a composite capture section that includes a first capture section 1540 and a second capture section 1560. Both the first and second capture sections 1540 and 1560 are attached to the intermediate portion 1524 of the coupling article 1520. The second capture section 1560 is located between the intermediate portion 1524 of the anchor section and, in one or more embodiments, may include an adhesive configured to secure the second capture section 1560 to a nasogastric tube. In one or more embodiments, the second capture section 1560 may be described as having a maximum lateral width measured transverse to the longitudinal direction D that is less than the maximum lateral width of the first capture section 1540 measured transverse to the longitudinal direction D.

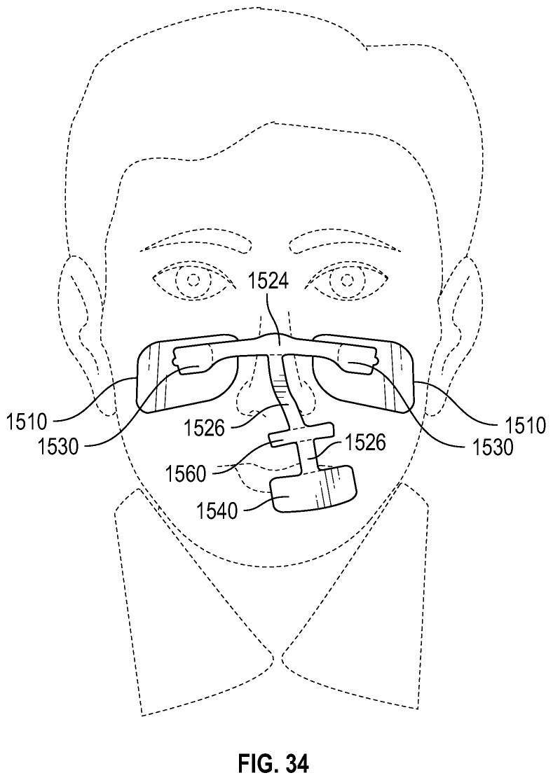

The illustrative embodiment of coupling article 1520 is depicted in FIG. 34 with the anchor sections 1530 attached to base articles 1510 on a patient's face. The intermediate portion 1524 spanning the anchor sections is positioned over a patient's nose while the longitudinal portion 1526 extends downward to support both the first capture section 1540 and the second capture section 1560. In one or more embodiments, the second capture section 1560 may preferably be positioned such that it may conveniently attach to a nasogastric tube extending out of a nostril of the patient's nose while the first capture section 1540 is positioned to capture an endotracheal tube inserted into the patient's mouth.

The endotracheal tube securement systems described herein may, in one or more embodiments, include one or more base articles and one or more coupling articles on a common delivery sheet as depicted in, e.g., FIGS. 1, 14, 17, and 26. In one or more embodiments, multiple common delivery sheets may be attached to each other, with each common delivery sheet containing an endotracheal tube securement system.

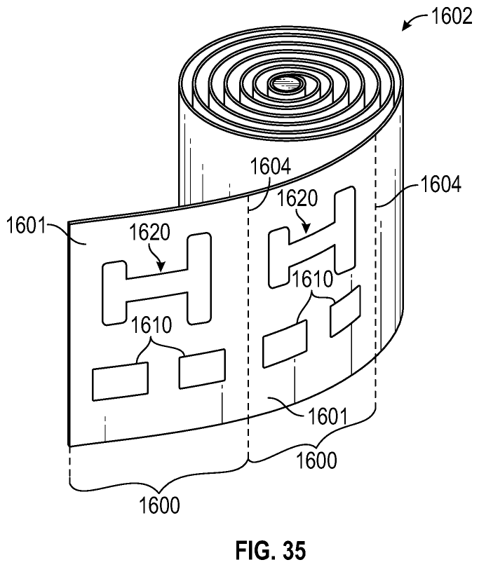

One illustrative embodiment of a roll-form delivery of multiple endotracheal tube securement systems on common delivery sheets is depicted in FIG. 35. The roll 1602 includes multiple common delivery sheets 1601, with each common delivery sheet 1601 carrying an endotracheal tube securement system 1600. One alternative embodiment in which a plurality of common delivery sheets carrying endotracheal tube securement systems as described herein may be arranged is in a fan-folded format. Other arrangements are also contemplated.

In one or more embodiments, adjacent common delivery sheets may be attached to each other along a line of separation 1604. The lines of separation 1604 may promote tearing or separation such that each of the common delivery sheets 1601 may be detached from the other common delivery sheets, e.g., from the roll 1602, for use of the endotracheal tube securement system located on the separated common delivery sheet 1601. In one or more embodiments, the line of separation may create a line of weakness in the material used in the delivery sheets 1601 (e.g., the line of weakness 1604 may include perforations, a thinned area, etc.) Alternatively, adjacent common delivery sheets 1601 may be separated from each other using scissors or a suitable cutting device.

In the depicted illustrative embodiment, each endotracheal tube securement system 1600 includes two base articles 1610 and a coupling article 1620, although alternative embodiments of endotracheal tube securement systems may include only one base article or three or more base articles, as well as two or more coupling articles.

### Methods

As discussed herein, endotracheal tube securement systems of the present disclosure include at least two parts or components: (i) one or more base articles that can be coupled (i.e., adhered) to skin, and (ii) one or more coupling articles having a capture section configured to secure the endotracheal tube and one or more anchor sections configured to be repositionably coupled to the one or more base articles to allow the endotracheal tube to be repositioned as desired without disrupting adhesion of the one or more base articles to skin, or requiring any portion of the one or more base articles to be removed. The one or more base articles can remain in position on the skin until it becomes necessary to change it or until the endotracheal tube is removed from the patient.

In one or more embodiments, methods of securing an endotracheal tube on a patient may include, e.g., adhesively attaching a base article on a patient using skin-contact adhesive on a first major surface of the base article. The method may further include securing a capture section of a coupling article to an endotracheal tube either before or after securing an anchor section of the coupling article to a first location on the base article, such that the endotracheal tube is attached to the first location on the base article through the coupling article. The method may further include, in one or more embodiments, removing the anchor section of the coupling article from the first location on the base article after securing the anchor section of the coupling article to the first location; and re-securing the anchor section of the coupling article to a second location different from the first location on the base article after removing the anchor section of the coupling article from the first location on the base article, wherein the endotracheal tube is attached to the second location on the base article through the coupling article.

In one or more embodiments of such a method, the capture section of the coupling article may remain secured to the endotracheal tube when removing the anchor section of the coupling article from the first location on the base article, and when re-securing the anchor section of the coupling article to the second location on the base article.

As discussed herein, in one or more embodiments, securing the anchor section of the coupling article to the base article comprises adhesively attaching the anchor section to the base article or securing the anchor section of the coupling article to the base article comprises attaching the anchor section to the base article using a mechanical fastener component.

One or more embodiments of another illustrative method of securing an endotracheal tube includes adhesively attaching a first base article on a patient using skin-contact adhesive on a first major surface of the first base article and adhesively attaching a second base article on a patient using skin-contact adhesive on a first major surface of the second base article. The method further includes securing a capture section of a coupling article to an endotracheal tube and securing a first anchor section of the coupling article to a first location on the first base article, wherein the endotracheal tube is attached to the first location on the first base article through the coupling article. The method further includes securing a second anchor section of the coupling article to a first location on the second base article, wherein the endotracheal tube is attached to the first location on the second base article through the coupling article. The method further includes removing the first anchor section of the coupling article from the first location on the first base article after securing the first anchor section of the coupling article to the first location on the first base article followed by re-securing the first anchor section of the coupling article to a second location different from the first location on the second base article after removing the first anchor section of the coupling article from the first location on the first base article, wherein the endotracheal tube is attached to the second location on the first base article through the coupling article. The method further includes removing the second anchor section of the coupling article from the first location on the second base article after securing the second anchor section of the coupling article to the first location on the second base article; followed by re-securing the second anchor section of the coupling article to a second location different from the first location on the second base article after removing the second anchor section of the coupling article from the first location on the second base article, wherein the endotracheal tube is attached to the second location on the second base article through the coupling article.

In one or more embodiments of such a method, the capture section of the coupling article remains secured to the endotracheal tube when removing the first and second anchor sections of the coupling article from the first locations on the first and second base articles, and when re-securing the first and second anchor sections of the coupling article to the second locations on the first and second base articles.

Securing the capture section of the coupling article to the endotracheal tube occurs before or after securing the first anchor section to the first location on the first base article.

In one or more embodiments, securing the first anchor section of the coupling article to the first base article comprises adhesively attaching the first anchor section to the first base article or attaching the first anchor section to the first base article using a mechanical fastener component.

### Backings

Suitable backings for base articles and coupling articles used in one or more embodiments of the endotracheal tube securement systems described herein can include, but are not limited to, one or more of a fabric, a woven fibrous web, a nonwoven fibrous web, a knit, a polymeric film, other familiar dressing materials, or combinations thereof. In some embodiments, the backings can include polymeric elastic films (e.g., transparent or non-transparent), and can include, but are not limited to, films formed of elastomeric polyurethanes, co-polyesters, polyethylenes, or combinations thereof The backings can be a high moisture vapor permeable film, i.e., a backing with a relatively high moisture vapor transmission rate (MVTR). U.S. Pat. No. 3,645,835 (Hodgson) describes methods of making such films and methods for testing their permeability. The backings can be constituted of natural or synthetic sources of raw materials.

In one or more embodiments, the backings of base articles and, optionally coupling articles, of one or more embodiments of endotracheal tube securement systems described herein may transmit moisture vapor at a rate equal to or greater than human skin. In embodiments in which the backing is coated with adhesive, the adhesive-coated backing may, in one or more embodiments, transmit moisture vapor at a rate of at least 300 g/m²/24 hrs/37° C./100-10% RH, and in one or more alternative embodiments, at least 700 g/m²/24 hrs/37° C./100-10% RH. The backings may, in one or more embodiments, be generally conformable to anatomical surfaces. As such, when the backing is applied to an anatomical surface, such as a nose, cheek, chin, etc., it conforms to the surface even when the surface moves or changes shape.

In one or more embodiments, the backings can be flexible. For example, the backings can be a film, paper, woven, knit, foam, nonwoven material, or a combination thereof, or one or more layers of film, paper, woven, knit, foam, nonwoven, or a combination thereof. In one or more embodiments, it may be desirable that at least a portion of the backing is formed of a transparent material to allow for viewing of underlying skin, a medical device, and/or a target site.

By way of example only, the backing of a base article of one or more embodiments of an endotracheal tube securement system described herein can be formed of a film available under the trade designation TEGADERM® from 3M Company, St. Paul, Minn.

The backing and skin-contact adhesive of one or more embodiments of base articles used in one or more embodiments of endotracheal tube securement systems described herein may be provided by a polyurethane stretchable nonwoven tape (e.g., 3M™ CoTran™ 9699 Melt Blow Polyurethane Tape from 3M Company, St. Paul, Minn.), any of the materials A-H of Table 1 below, other suitable tapes/backings, or a combination thereof.

The backings used in one or more embodiments of coupling articles of one or more embodiments of endotracheal tube securement systems described herein may be formed of, e.g., one or more of the backings described in I-N of Table 1 below, a combination of one or more of the backings described in I-N of Table 1 in combination with one or more the backings of A-H in Table 1. Many other suitable tapes/backings could also be used for the coupling articles described herein.

TABLE 1
Exemplary Backing and Adhesive Combinations

| Material | Description | Source |
|---|---|---|
| A-Foam Tape | 3M ™ Polyethylene Foam Medical Tape 1774W, | 3M Company, |
| | 510 micron, closed cell, polyethylene foam | St. Paul, MN |
| | backing, coated with 60 micron thick pressure | |
| | sensitive acrylate adhesive. | |
| B-PU-NW Tape | 3M ™ CoTran ™ 9699 Melt Blown Polyurethane | 3M Company, |
| | Tape: 254 micron thick polyurethane/polyethylene | St. Paul, MN |
| | backing coated with a gentle medical acrylate | |
| | adhesive | |
| C-PU Film Tape | 3M ™ Polyurethane Tape 9834; 20 micron | 3M Company, |
| | polyurethane film with 25 micron thick gentle | St. Paul, MN |
| | medical acrylate adhesive | |
| D-coPET Film | 3M ™ Steri-Drape ™ 2 incise drape: | 3M Company, |
| Drape | 25 micron thick elastomeric copolyester | St. Paul, MN |
| | backing coated with 51 micron thick | |
| | pressure sensitive acrylate adhesive. | |
| E-Si Film | SILPURAN ™ Film 2030; medical grade silicone | Wacker Chemie AG, |
| | film, 100 micron thick | Munich Germany |
| F-PE Tape | 3M ™ Blenderm ™ Surgical Tape 1525 - | 3M Company, |
| | Polyethylene backing coated with gentle medical | St. Paul, MN |
| | acrylate adhesive | |
| G-LDPE Film | 3M ™ Steri-Drape ™ incise drape: 30 micron | 3M Company, |
| Drape | thick low density polyethylene backing coated | St. Paul, MN |
| | with 51 micron thick pressure sensitive acrylate | |
| | adhesive | |
| H-coPET-AM | 3M ™ Ioban ™ 2 Antimicrobial Incise Drape; 25 | 3M Company, |
| Drape | micron thick elastomeric copolyester backing | St. Paul, MN |
| | coated with 51 micron thick iodophor impregnated | |
| | (antimicrobial) pressure sensitive acrylate adhesive | |
| I-PET-NW Tape | 3M ™ Spunlaced Polyester Nonwoven | 3M Company, |
| | Medical Tape 1776: polyester backing, coated | St. Paul, MN |
| | with a medical, pressure sensitive acrylate | |
| | adhesive | |
| J-RA-NW Tape | 3M ™ Rayon Acetate Woven Medical Tape 1538; | 3M Company, |
| | Rayon acetate woven cloth backing coated with a | St. Paul, MN |
| | medical, pressure sensitive acrylate adhesive | |
| K-RA-MP NW | 3M ™ MICROPORE 1530 Surgical Tape; | 3M Company, |
| Tape | microporous rayon nonwoven backing coated with | St. Paul, MN |
| | a medical, pressure sensitive acrylate adhesive | |
| L-CAT Tape | 3M ™ Cloth Adhesive Tape 2950; high strength | 3M Company, |
| | cotton backing coated with a medical, pressure | St. Paul, MN |
| | sensitive acrylate adhesive | |
| M-NW fiber strip | 3M ™ Steri-Strip Skin Closure 1548; nonwoven | 3M Company, |
| | backing, fiber reinforced with a medical, pressure | St. Paul, MN |
| | sensitive acrylate adhesive | |
| N-Fabric tape | 3M ™ Multipore dry: fabric backing, pressure | 3M Company, |
| | sensitive acrylate adhesive | St. Paul, MN |

### Release Liners

Release liners suitable for use with the systems of the present disclosure (e.g., as all or a portion of a common delivery sheet) can include, but are not limited to, kraft papers, polyethylene, embossed polyethylene, polypropylene, polyester, or combinations thereof. Such liners can be coated with release agents, such as fluorochemicals, silicones, or other suitable low surface energy materials. Other adhesives and release liner combinations known to those of ordinary skill in the art can also be employed in the systems of the present disclosure. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films, commercially available from H. P. Smith Co., and fluoropolymer coated polyester films, commercially available from 3M Company (St. Paul) under the brand “SCOTCHPAK™” release liners.

### Adhesives

As described herein, adhesives used in one or more anchor sections to attach the coupling articles to base articles of endotracheal tube securement systems or to other components in the coupling articles themselves (e.g., mechanical fastener material or another layer) can be described as securing adhesives. Such securing adhesives may have an adhesion that is higher than the skin-contact adhesives used to adhesively attach the base articles to patient skin (e.g., the skin-contact adhesive 114 in FIG. 4 or 114′ in FIG. 5). Adhesive 132 in FIG. 3B is one example of a securing adhesive that may be used as a means for repositionably attaching an anchor section of a coupling article to a base article as described herein. The use of release layers, etc. on the base articles can, however, allow for removal of the anchor sections from base articles while allowing the base articles to remain attached to the patient's skin despite the higher adhesion of the securing adhesives on the anchor sections.

Similarly, the adhesives used in the capture sections of the coupling articles described herein, which are configured to secure the capture section to an endotracheal tube, may also be described as securing adhesives and may also have an adhesion that is higher than the skin-contact adhesives used to adhesively attach the base articles to patient skin. Adhesive 142 in FIG. 4 is one example of a securing adhesive that may be used to attach a capture section of a coupling article to an endotracheal tube as described herein.

In some embodiments, the securing adhesives and the skin-contact adhesives may be of the same or similar classes of adhesive, but have different adhesion levels. For example, the securing adhesive and/or the skin-contact adhesive may be an acrylate, silicone, urethane, hydrogel, hydrocolloid, natural rubber, or synthetic rubber. Adhesion can also be tuned through changes in adhesive composition, adhesive thickness, or adhesive surface area (e.g., by employing a pattern-coated adhesive).

“Adhesion” refers to the force required to separate an adhesive from an underlying substrate. Adhesion can be measured in a number of ways. For example, adhesion can be defined by peel force or shear force. In some embodiments, adhesion can be defined by peel adhesion using ASTM D3330/D3330M-04 (2010). In some embodiments, adhesion can be defined by shear adhesion using ASTM D3654M-06 (2011). Adhesion is dependent on the specific substrate being adhered to, as well as the time the pressure-sensitive adhesive (PSA) is allowed to dwell on the substrate.

For example, typical peel adhesion values exhibited by pressure-sensitive adhesives in medical dressings maybe in the range of 20 to 300 g/cm as measured from stainless steel. In some embodiments, at least 10% higher peel adhesion, as measured by ASTM D3330/D3330M-04 (2010), of the securing adhesive over the skin-contact adhesive may realize the benefit of both securing to base article and, optionally a catheter, while providing gentle adhesion to the skin.

In some embodiments, the securing adhesive can be an acrylate adhesive and the skin-contact adhesive can be a silicone adhesive. The term “acrylate” or “acrylate-based” or “acrylate-containing” refers to monomeric acrylic or methacrylic esters of alcohols. Acrylate and methacrylate monomers are referred to collectively herein as “acrylate” monomers. Materials that are described as “acrylate-based” or “acrylate-containing” contain at least some acrylate monomers and may contain additional co-monomers.

Acrylate adhesives may be used for securing articles to each other (e.g., coupling articles to base articles and/or coupling articles to catheters) or, optionally, for securing the base articles to skin. The adhesion can be manipulated to have high adhesion or low adhesion. Generally, the adhesion between acrylate adhesives and another material will increase over time.

Examples of potentially suitable skin-contact pressure sensitive adhesives (PSAs) include rubber based adhesives (e.g., tackified natural rubbers, synthetic rubbers, and styrene block copolymers), acrylics (e.g., polymerized (meth)acrylates), poly(alpha-olefins), polyurethanes, and silicones. Amine containing polymers can also be used which have amine groups in the backbone, pendant thereof, or combinations thereof. A suitable example includes a poly(ethyleneimine).

Useful skin-contact adhesives can be any of those that are compatible with skin and useful for wound dressings, such as those disclosed in U.S. Pat. No. Re. 24,906 (Ulrich), U.S. Pat. No. 5,849,325 (Heinecke et al.), and U.S. Pat. No. 4,871,812 (Lucast et. al.) (water-based and solvent-based adhesives); U.S. Pat. No. 4,833,179 (Young et al.) (hot-melt adhesives); U.S. Pat. No. 5,908,693 (Delgado et al.) (microsphere adhesives); U.S. Pat. Nos. 6,171,985 and 6,083,856 (both to Joseph et al.) (low trauma fibrous adhesives); and, U.S. Pat. No. 6,198,016 (Lucast et al.), U.S. Pat. No. 6,518,343 (Lucast et al.), and U.S. Pat. No. 6,441,092 (Gieselman) (wet-skin adhesives). Inclusion of medicaments or antimicrobial agents in the adhesive is also contemplated, as described in U.S. Pat. No. 4,310,509 (Berglund) and U.S. Pat. No. 4,323,557 (Rosso).

Silicone and acrylic based pressure sensitive adhesives are commonly utilized for adhering to the skin.

Silicone PSAs typically include two major components, a polymer or gum, and a tackifying resin. The polymer is typically a high molecular weight polydimethylsiloxane or polydimethyl-diphenylsiloxane, that contains residual silanol functionality (SiOH) on the ends of the polymer chain, or a block copolymer including polydiorganosiloxane soft segments and urea terminated hard segments. The tackifying resin is generally a three-dimensional silicate structure that is endcapped with trimethylsiloxy groups (OSiMe₃) and also contains some residual silanol functionality. Examples of tackifying resins include SR 545, from General Electric Co., Silicone Resins Division, Waterford, N.Y., and MQD-32-2 from Shin-Etsu Silicones of America, Inc., Torrance, Calif. Manufacture of typical silicone PSAs is described in U.S. Pat. No. 2,736,721 (Dexter). Manufacture of silicone urea block copolymer PSA is described in U.S. Pat. No. 5,214,119 (Leir et al.). In some embodiments, the silicone adhesive may be characterized as gentle to skin such as described in U.S. Pat. No. 8,541,481 (Determan et al.), U.S. Patent Publication No. US2013/0040073 (Pett et al.), U.S. Pat. No. 7,407,709 (Zhou et al.) and U.S. Pat. No. 7,807,268 (Zhou et al.). Examples of suitable silicone adhesive systems can include, but are not limited to, products available under the following trade designations: Dow Corning MG 7-9850, Wacker SILPURAN® 2110 and 2130, Bluestar SILBIONE® RT Gel 4317 and 4320, Nusil MED-6345 and 6350.

Acrylic adhesive typically comprise a copolymer of at least one C₄-C₁₂ alkyl (meth)acrylate such as isooctyl acrylate or 2-ethylehexylacrylate and at least one high Tg (e.g. polar) comonomer such as (meth)acrylamide, N-vinyl pyrrolidone, poly(ethylene oxide)acrylate, and mixture thereof. In typical embodiments, the acrylic adhesive comprises at least 90 wt.-% C₄-C₁₂ alkyl (meth)acrylate(s). Suitable examples include a 90:10 isooctyl acrylate: acrylic acid copolymer, a 70:15:15 isooctyl acrylate: ethylene oxide acrylate: acrylic acid terpolymer, and a 25:69:6 2-ethylhexylacrylate: butyl acrylate: acrylic acid terpolymer. Another acrylic adhesive composition includes a 97:3 isooctyl acrylate: acrylamide copolymer 65:15:20 2-ethylhexylacrylate: acrylic acid: copolymer blended with a nonreactive polyalkylene oxide copolymer under the trade designation PLURONIC. Additional useful adhesives are described in U.S. Pat. No. 3,389,827 (Abere et al.), U.S. Pat. No. 4,112,213 (Waldman), U.S. Pat. No. 4,310,509 (Berglund et al.), and U.S. Pat. No. 4,323,557 (Rosso et al.).

For skin-contact adhesives, it is desirable that the adhesive is able to transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as perforating the adhesive or pattern coating the adhesive, as described in U.S. Pat. No. 4,595,001 (Potter et al.) and U.S. Patent Application. Publication No. 2008-0233348 (also U.S. Pat. No. 7,947,366) (Ishiwatari et al.). Each of the securing or skin-contact adhesive can optionally be applied in a discontinuous manner.

### Embodiments

The following numbered embodiments are intended to be illustrative of the present disclosure and not limiting.

1. An endotracheal tube securement system, the system comprising:

a base article configured to be adhered to a human face, the base article having a first major surface comprising a skin-contact adhesive and a second major surface opposite the first major surface; and

a coupling article comprising a first major surface and a second major surface, wherein the coupling article comprises an anchor section and a capture section arranged along a longitudinal direction;

wherein the first major surface of the coupling article occupied by the anchor section comprises means for repositionably attaching the anchor section to the second major surface of the base article;

wherein the first major surface of the coupling article occupied by the capture section comprises adhesive configured to secure the capture section to an endotracheal tube;

and wherein the anchor section comprises a maximum lateral width measured transverse to the longitudinal direction that is greater than a maximum lateral width of the capture section.

2. The system of embodiment 1, wherein the base article and the coupling article are attached to a common delivery sheet.
3. The system of embodiment 2, wherein the common delivery sheet is attached to a plurality of common delivery sheets, wherein each common delivery sheet of the plurality of delivery sheets carries another endotracheal tube securement system.
4. The system of any one of embodiments 1 to 3, wherein the first major surface of the coupling article occupied by the anchor section of the coupling article is smaller than the second major surface of the base article.
5. The system of any one of embodiments 1 to 4, wherein the maximum lateral width of the anchor section is two or more times greater than the maximum lateral width of the capture section.
6. The system of any one of embodiments 1 to 5, wherein the means for repositionably attaching the anchor section to the second major surface of the base article comprises a first attachment area and a second attachment area, wherein the first and second attachment areas occupy separate and discrete portions of the first major surface of the coupling article within the anchor section, and wherein the maximum lateral width of the anchor section is measured between a geometric center of the first attachment area and a geometric center of the second attachment area.
7. The system of embodiment 6, wherein a distance from the geometric center of the first attachment area to a geometric center of the capture section is less than the distance from the geometric center of the first attachment area to the geometric center of the second attachment area.
8. The system of embodiment 7, wherein a distance from the geometric center of the first attachment area to a geometric center of the capture section is less than the distance from the geometric center of the first attachment area to the geometric center of the second attachment area.
9. The system of any one of embodiments 6 to 8, wherein the first attachment area is smaller than the second major surface of the base article.
10. The system of embodiment 9, wherein the second attachment area is smaller than the second major surface of the base article.
11. The system of any one of embodiments 6 to 8, wherein the base article comprises a first base article and wherein the system further comprises a second base article, wherein the second base article is configured to be adhered to a human face, the second base article having a first major surface comprising a skin-contact adhesive and a second major surface opposite the first major surface.
12. The system of embodiment 11, wherein the second base article has the same construction as the first base article.
13. The system of any one of embodiments 11 to 12, wherein the first and second base articles and the coupling article are attached to a common delivery sheet.
14. The system of embodiment 13, wherein the common delivery sheet is attached to a plurality of common delivery sheets, wherein each common delivery sheet of the plurality of delivery sheets carries another endotracheal tube securement system
15. The system of any one of embodiments 11 to 14, wherein the first attachment area is smaller than the second major surface of the first base article, and wherein the second attachment area is smaller than the second major surface of the second base article.
16. The system of any one of embodiments 1 to 15, wherein the capture section comprises a first capture section and wherein the coupling article comprises a second capture section, wherein the second capture section is located between the anchor section and the first capture section, and wherein the first major surface of the coupling article occupied by the second capture section comprises adhesive configured to secure the second capture section to a nasogastric tube.
17. The system of embodiment 16, wherein the second capture section comprises a maximum lateral width measured transverse to the longitudinal direction that is less than the maximum lateral width of the first capture section.
18. The system of any one of embodiments 1 to 17, wherein the means for repositionably attaching the anchor section to the second major surface of the base article comprises adhesive located on the first major surface of the coupling article occupied by the anchor section.
19. The system of embodiment 18, wherein the means for repositionably attaching the anchor section to the second major surface of the base article comprises a release layer on the second major surface of the base article.
20. The system of any one of embodiments 1 to 17, wherein the means for repositionably attaching the anchor section to the second major surface of the base article comprises:

a first mechanical fastener component on the first major surface of the coupling article; and

a second mechanical fastener component on the second major surface of the base article, wherein the first and second mechanical fastener components are configured to attach to each other.

21. The system of any one of embodiments 1 to 17, wherein the means for repositionably attaching the anchor section to the second major surface of the base article comprises a layer of non-tacky/cohesive polymeric material on the first major surface of the coupling article occupied by the anchor section and/or the second major surface of the base article.
22. An endotracheal tube securement system, the system comprising:

a first base article configured to be adhered to a human face, the first base article having a first major surface comprising a skin-contact adhesive and a second major surface opposite the first major surface;

a second base article configured to be adhered to a human face, the second base article having a first major surface comprising a skin-contact adhesive and a second major surface opposite the first major surface; and

a coupling article comprising a first major surface and a second major surface, wherein the coupling article comprises a first anchor section, a second anchor section, and a capture section, wherein the first anchor section and the second anchor section are separate and discrete areas that are separated from each other along a lateral direction, and wherein the capture section is separated from both the first anchor section and the second anchor section along a longitudinal direction that is transverse to the lateral direction;

wherein the first major surface of the coupling article occupied by the first anchor section comprises means for repositionably attaching the anchor section to the second major surface of the first base article;

wherein the first major surface of the coupling article occupied by the second anchor section comprises means for repositionably attaching the second anchor section to the second major surface of the second base article;

and wherein the first major surface of the coupling article occupied by the capture section comprises adhesive configured to secure the capture section to an endotracheal tube.

23. The system of embodiment 22, wherein the first and second base articles and the coupling article are attached to a common delivery sheet.
24. The system of embodiment 23, wherein the common delivery sheet is attached to a plurality of common delivery sheets, wherein each common delivery sheet of the plurality of delivery sheets carries another endotracheal tube securement system.
25. The system of any one of embodiments 22 to 24, wherein the first major surface of the coupling article occupied by the first anchor section of the coupling article is smaller than the second major surface of the first base article.
26. The system of embodiment 25, wherein the first major surface of the coupling article occupied by the second anchor section of the coupling article is smaller than the second major surface of the second base article.
27. The system of any one of embodiments 22 to 26, wherein a geometric center of the first anchor section is spaced apart from a geometric center of the second anchor section by a distance that is greater than a maximum lateral width of the capture section as measured along the lateral direction.
28. The system of any one of embodiments 22 to 27, wherein a distance from the geometric center of the first anchor section to a geometric center of the capture section is less than the distance from the geometric center of the first anchor section to the geometric center of the second anchor section.
29. The system of embodiment 28, wherein a distance from the geometric center of the second anchor section to the geometric center of the capture section is less than the distance from the geometric center of the first anchor section to the geometric center of the second anchor section.
30. The system of any one of embodiments 22 to 29, wherein the means for repositionably attaching the first anchor section to the second major surface of the first base article comprises adhesive located on the first major surface of the coupling article occupied by the first anchor section, and wherein the means for repositionably attaching the second anchor section to the second major surface of the second base article comprises adhesive located on the first major surface of the coupling article occupied by the second anchor section.
31. The system of embodiment 30, wherein the means for repositionably attaching the first anchor section to the second major surface of the first base article comprises a release layer on the second major surface of the first base article, and wherein the means for repositionably attaching the second anchor section to the second major surface of the second base article comprises a release layer on the second major surface of the second base article.
32. The system of any one of embodiments 22 to 29, wherein the means for repositionably attaching the first anchor section to the second major surface of the first base article comprises:

a first mechanical fastener component on the first major surface of the coupling article occupied by the first anchor section; and

a second mechanical fastener component on the second major surface of the first base article, wherein the first and second mechanical fastener components are configured to attach to each other;

and wherein the means for repositionably attaching the second anchor section to the second major surface of the second base article comprises:

a third mechanical fastener component on the first major surface of the coupling article occupied by the second anchor section; and

a fourth mechanical fastener component on the second major surface of the second base article, wherein the third and fourth mechanical fastener components are configured to attach to each other.

33. The system of any one of embodiments 22 to 29, wherein the means for repositionably attaching the first and second anchor sections to the second major surface of the base article comprises a layer of non-tacky/cohesive polymeric material on the first major surface of the coupling article occupied by the first and second anchor sections and/or the second major surfaces of the first and second base articles.
34. An endotracheal tube securement system, the system comprising:

a base article configured to be adhered to a human face, the base article having a first major surface comprising a skin-contact adhesive and a second major surface opposite the first major surface; and

a first coupling article and a second coupling article, wherein each of the first and second coupling articles comprises:

a first major surface and a second major surface, wherein each of the first and second coupling articles comprises an anchor section and a capture section arranged along a longitudinal direction;

wherein the first major surface occupied by the anchor section of each of the first and second coupling articles comprises means for repositionably attaching the anchor section to the second major surface of the base article;

and wherein the first major surface occupied by the capture section of each of the first and second coupling articles comprises adhesive configured to secure the capture section to an endotracheal tube.

35. The system of embodiment 34, wherein the base article and each of the first and second coupling articles are attached to a common delivery sheet.
36. The system of embodiment 35, wherein the common delivery sheet is attached to a plurality of common delivery sheets, wherein each common delivery sheet of the plurality of delivery sheets carries another endotracheal tube securement system.
37. The system of any one of embodiments 34 to 36, wherein the anchor section of each of the first and second coupling articles comprises a maximum lateral width measured transverse to the longitudinal direction that is greater than a maximum lateral width of the capture section.
38. The system of any one of embodiments 34 to 36, wherein the maximum lateral width of the anchor section of each of the first and second coupling articles is two or more times greater than the maximum lateral width of the capture section.
39. The system of any one of embodiments 34 to 38, wherein the first major surface of occupied by the anchor section of each of the first and second coupling articles is smaller than the second major surface of the base article.
40. The system of any one of embodiments 34 to 39, wherein the base article comprises a first base article and wherein the system further comprises a second base article, wherein the second base article is configured to be adhered to a human face, the second base article having a first major surface comprising a skin-contact adhesive and a second major surface opposite the first major surface.
41. The system of embodiment 40, wherein the second base article has the same construction as the first base article.
42. The system of any one of embodiments 40 to 41, wherein the first and second base articles and the coupling article are attached to a common delivery sheet.
43. The system of embodiment 42, wherein the common delivery sheet is attached to a plurality of common delivery sheets, wherein each common delivery sheet of the plurality of delivery sheets carries another endotracheal tube securement system.
44. The system of any one of embodiments 34 to 43, wherein the means for repositionably attaching the anchor section of each of the first and second coupling articles to the second major surface of the base article comprises adhesive located on the first major surface of the coupling article occupied by the anchor section.
45. The system of embodiment 44, wherein the means for repositionably attaching the anchor section to the second major surface of the base article comprises a release layer on the second major surface of the base article.
46. The system of any one of embodiments 34 to 43, wherein the means for repositionably attaching the anchor section of each of the first and second coupling articles to the second major surface of the base article comprises:

a first mechanical fastener component on the first major surface of the coupling article; and

a second mechanical fastener component on the second major surface of the base article, wherein the first and second mechanical fastener components are configured to attach to each other.

47. The system of any one of embodiments 34 to 43, wherein the means for repositionably attaching the anchor sections of the first and second coupling articles to the second major surface of the base article comprises a layer of non-tacky/cohesive polymeric material on the first major surfaces occupied by the anchor sections of each of the first and second coupling articles and/or the second major surface of the base article.
48. An endotracheal tube securement system, the system comprising:

a base article configured to be adhered to a human face, the base article having a first major surface comprising a skin-contact adhesive and a second major surface opposite the first major surface; and

a coupling article comprising a first major surface and a second major surface, wherein the coupling article comprises a first anchor section, a second anchor section, and a capture section, wherein the first anchor section and the second anchor section are separate and discrete areas that are separated from each other along a lateral direction, and wherein the capture section is located between the first anchor section and the second anchor section;

wherein the first major surface of the coupling article occupied by the first anchor section comprises means for repositionably attaching the anchor section to the second major surface of the base article;

wherein the first major surface of the coupling article occupied by the second anchor section comprises means for repositionably attaching the second anchor section to the second major surface of the base article;

and wherein the first major surface of the coupling article occupied by the capture section comprises adhesive configured to secure the capture section to an endotracheal tube.

49. The system of embodiment 48, wherein the capture section comprises a first leg attached to the first anchor section and terminating proximate the second anchor section and a second leg attached to the second anchor section and terminating proximate the first anchor section.
50. The system of embodiment 49, a first portion of the adhesive of the capture section is located on the first leg and a second portion of the adhesive of the capture section is located on the second leg.
51. The system of any one of embodiments 48 to 50, wherein the base article and the coupling article are attached to a common delivery sheet.
52. The system of embodiment 51, wherein the common delivery sheet is attached to a plurality of common delivery sheets, wherein each common delivery sheet of the plurality of delivery sheets carries another endotracheal tube securement system.
53. The system of any one of embodiments 48 to 52, wherein the first major surface of the coupling article occupied by the first anchor section of the coupling article is smaller than the second major surface of the first base article.
54. The system of embodiment 53, wherein the first major surface of the coupling article occupied by the second anchor section of the coupling article is smaller than the second major surface of the base article.
55. The system of any one of embodiments 48 to 54, wherein a geometric center of the first anchor section is spaced apart from a geometric center of the second anchor section by a distance that is greater than a maximum lateral width of the capture section as measured along the lateral direction.
56. The system of any one of embodiments 48 to 54, wherein a distance from the geometric center of the first anchor section to a geometric center of the capture section is less than the distance from the geometric center of the first anchor section to the geometric center of the second anchor section.
57. The system of embodiment 56, wherein a distance from the geometric center of the second anchor section to the geometric center of the capture section is less than the distance from the geometric center of the first anchor section to the geometric center of the second anchor section.
58. The system of any one of embodiments 48 to 57, wherein the base article comprises a first base article and wherein the system further comprises a second base article, wherein the second base article is configured to be adhered to a human face, the second base article having a first major surface comprising a skin-contact adhesive and a second major surface opposite the first major surface.
59. The system of embodiment 58, wherein the second base article has the same construction as the first base article.
60. The system of any one of embodiments 58 to 59, wherein the first and second base articles and the coupling article are attached to a common delivery sheet.
61. The system of embodiment 60, wherein the common delivery sheet is attached to a plurality of common delivery sheets, wherein each common delivery sheet of the plurality of delivery sheets carries another endotracheal tube securement system.
62. The system of any one of embodiments 58 to 61, wherein the first anchor section is smaller than the second major surface of the first base article, and wherein the second anchor section is smaller than the second major surface of the second base article.
63. The system of any one of embodiments 48 to 62, wherein the means for repositionably attaching the first anchor section to the second major surface of the base article comprises adhesive located on the first major surface of the coupling article occupied by the first anchor section.
64. The system of embodiment 63, wherein the means for repositionably attaching the first anchor section to the second major surface of the base article comprises a release layer on the second major surface of the base article.
65. The system of any one of embodiments 48 to 62, wherein the means for repositionably attaching the first anchor section to the second major surface of the base article comprises:

a first mechanical fastener component on the first major surface of the coupling article; and

a second mechanical fastener component on the second major surface of the base article, wherein the first and second mechanical fastener components are configured to attach to each other.

66. The system of any one of embodiments 48 to 62, wherein the means for repositionably attaching the first and second anchor sections to the second major surface of the base article comprises a layer of non-tacky/cohesive polymeric material on the first major surface of the coupling article occupied by the first and second anchor sections and/or the second major surface of the base article.
67. The system of any one of embodiments 1 to 66, wherein at least a portion of the first major surface of at least one coupling article of the system comprises a hydrocolloid coating.
68. The system of any of embodiments 1 to 67, wherein the base article has a percent elongation of at least 200%, and wherein the coupling article has a percent elongation of no greater than 50%.
69. The system of any of embodiments 1 to 67, wherein the base article comprises a backing formed of a stretch release material.
70. A method of securing an endotracheal tube, the method comprising:

adhesively attaching a base article on a patient using skin-contact adhesive on a first major surface of the base article;

securing a capture section of a coupling article to an endotracheal tube;

securing an anchor section of the coupling article to a first location on the base article, wherein the endotracheal tube is attached to the first location on the base article through the coupling article;

removing the anchor section of the coupling article from the first location on the base article after securing the anchor section of the coupling article to the first location; and

re-securing the anchor section of the coupling article to a second location different from the first location on the base article after removing the anchor section of the coupling article from the first location on the base article, wherein the endotracheal tube is attached to the second location on the base article through the coupling article.

71. The method of embodiment 70, wherein the capture section of the coupling article remains secured to the endotracheal tube when removing the anchor section of the coupling article from the first location on the base article, and when re-securing the anchor section of the coupling article to the second location on the base article.
72. The method of any one of embodiments 70 to 71, wherein securing the capture section of the coupling article to the endotracheal tube occurs before securing the anchor section to the first location on the base article.
73. The method of any one of embodiments 70 to 71, wherein securing the capture section of the coupling article to the endotracheal tube after before securing the anchor section to the first location on the base article.
74. The method of any one of embodiments 70 to 73, wherein securing the anchor section of the coupling article to the base article comprises adhesively attaching the anchor section to the base article.
75. The method of any one of embodiments 70 to 73, wherein securing the anchor section of the coupling article to the base article comprises attaching the anchor section to the base article using a mechanical fastener component.
76. A method of securing an endotracheal tube, the method comprising:

adhesively attaching a first base article on a patient using skin-contact adhesive on a first major surface of the first base article;

adhesively attaching a second base article on a patient using skin-contact adhesive on a first major surface of the second base article;

securing a capture section of a coupling article to an endotracheal tube;

securing a first anchor section of the coupling article to a first location on the first base article, wherein the endotracheal tube is attached to the first location on the first base article through the coupling article;

securing a second anchor section of the coupling article to a first location on the second base article, wherein the endotracheal tube is attached to the first location on the second base article through the coupling article;

removing the first anchor section of the coupling article from the first location on the first base article after securing the first anchor section of the coupling article to the first location on the first base article;

re-securing the first anchor section of the coupling article to a second location different from the first location on the second base article after removing the first anchor section of the coupling article from the first location on the first base article, wherein the endotracheal tube is attached to the second location on the first base article through the coupling article;

removing the second anchor section of the coupling article from the first location on the second base article after securing the second anchor section of the coupling article to the first location on the second base article; and

re-securing the second anchor section of the coupling article to a second location different from the first location on the second base article after removing the second anchor section of the coupling article from the first location on the second base article, wherein the endotracheal tube is attached to the second location on the second base article through the coupling article.

77. The method of embodiment 76, wherein the capture section of the coupling article remains secured to the endotracheal tube when removing the first and second anchor sections of the coupling article from the first locations on the first and second base articles, and when re-securing the first and second anchor sections of the coupling article to the second locations on the first and second base articles.
78. The method of any one of embodiments 76 to 77, wherein securing the capture section of the coupling article to the endotracheal tube occurs before securing the first anchor section to the first location on the first base article.
79. The method of any one of embodiments 76 to 77, wherein securing the capture section of the coupling article to the endotracheal tube after before securing the first anchor section to the first location on the first base article.
80. The method of any one of embodiments 76 to 79, wherein securing the first anchor section of the coupling article to the first base article comprises adhesively attaching the first anchor section to the first base article.
81. The method of any one of embodiments 76 to 79, wherein securing the first anchor section of the coupling article to the first base article comprises attaching the first anchor section to the first base article using a mechanical fastener component.

Each embodiment shown in the figures is illustrated as a separate embodiment for clarity in illustrating a variety of features of the endotracheal tube securement systems of the present disclosure. However, it should be understood that any combination of elements and features of any of the embodiments illustrated in the figures and described herein can be employed in the endotracheal tube securement systems described herein.

In addition, various features and elements described in connection with the nasogastric tube securement systems described in the patent applications identified below may be used in one or more embodiments of the endotracheal tube securement systems described herein. For example, one or more embodiments of the endotracheal tube securement systems described herein may include one or more components of the nasogastric tube securement systems described in the applications identified below, e.g., one or more of the described coupling layers, base layers, etc. of the nasogastric securement systems may be included with the endotracheal tube securement systems described herein (e.g., on a common delivery sheet with the coupling article(s) and base article(s) of an endotracheal tube securement system). The applications include: PCT Application No. US2016/047489 (which claims priority to U.S. Provisional Application No. 62/208055 (Attorney Docket No.

76503US002)); PCT Application No. US2016/047491 (which claims priority to U.S. Provisional Application No. 62/208058 (Attorney Docket No. 76855US002)); PCT Application No. US2016/047494 (which claims priority to U.S. Provisional Application No. 62/208060 (Attorney Docket No. 76856US002)); PCT Application No. US2016/047495 (which claims priority to U.S. Provisional Application No. 62/208065 (Attorney Docket No. 76857US002)); and PCT Application No. US2016/047500 (which claims priority to U.S. Provisional Application No. 62/208069 (Attorney Docket No. 76858US002)). Each of the above-identified PCT applications was filed on Aug. 18, 2016 with the title: “Nasogastric Tube Securement Systems and Methods of Using Same” and each of the above-identified U.S. Provisional applications was filed on Aug. 21, 2015 with that same title.

### Test Methods

### Tensile Test Method

Percent elongation is measured using a Universal test machine available from Kratos Industrial Equipment Ltda., BR, model K2000MP with a load cell of 20 kgf (196 N), depending on the properties of the backing to be tested, and with the gauge distance and the kart speed set according to the backing characteristics, as set forth in Table 2 below.

TABLE 2
Gauge Distance and Test Speed for Elongation Testing

| Conditions | Distance between gauges | Test speed |
|---|---|---|
| <100% Elongation | 100 mm | 100 mm/min |
| between 100-400% | 50 mm | 200 mm/min |
| >400% | 20 mm | 200 mm/min |

The complete disclosure of the patents, patent documents, and publications identified herein are incorporated by reference in their entirety as if each were individually incorporated. To the extent there is a conflict or discrepancy between this document and the disclosure in any such incorporated document, this document will control.

The illustrative embodiments described above and depicted in the figures are presented by way of example only and are not intended as a limitation upon the concepts and principles of the present disclosure. As such, it will be appreciated by one having ordinary skill in the art that various changes in the elements and their configuration and arrangement are possible without departing from the scope of the present disclosure.

From the above disclosure of the general principles of the present invention and the preceding detailed description, those skilled in this art will readily comprehend the various modifications, re-arrangements and substitutions to which the present invention is susceptible, as well as the various advantages and benefits the present invention may provide. Therefore, the scope of the invention should be limited only by the following claims and equivalents thereof. In addition, it is understood to be within the scope of the present invention that the disclosed and claimed systems, kits and methods may be useful in other applications. Therefore, the scope of the invention may be broadened to include the use of the claimed and disclosed methods for such other applications.

What is claimed is:

1. An tube securement system comprising:
a coupling article comprising a backing layer with first major surface and a second major surface, wherein the coupling article comprises an anchor section, a capture section, and a longitudinal portion extending along a longitudinal direction separating the anchor section from the capture section;
a first adhesive on the first major surface of the backing at the anchor section;
a second adhesive on the first major surface of the backing at the capture section;
wherein the first major surface of the backing at the longitudinal portion is free of adhesive.

2. The tube securement device of claim 1, wherein the anchor section comprises a maximum lateral width measured transverse to the longitudinal direction that is greater than a maximum lateral width of the capture section.

3. The tube securement device of claim 1, wherein the anchor section extends laterally from the longitudinal portion.

4. The tube securement device of claim 1, wherein the capture section extends laterally from the longitudinal portion.

5. The tube securement device of claim 1, wherein the anchor section comprises a first anchor section and a second anchor section that are separate and discrete areas that are separated from each other along a lateral direction.

6. The tube securement device of claim 5, wherein the capture section is separated from both the first anchor section and the second anchor section along the longitudinal direction.

7. The tube securement device of claim 1, wherein the capture section comprises a first capture section and a second capture section.

8. The tube securement system, further comprising:
a base article configured to be adhered to a human face, the base article having a first major surface comprising a skin-contact adhesive and a second major surface opposite the first major surface; and

9. The tube securement system of claim 8, wherein the base article and the coupling article are attached to a common delivery sheet.

10. The tube securement device of claim 1, further comprising:
a tube;
wherein, in use, second adhesive secures to the tube is secured to the first adhesive;
wherein, in use, the first adhesive secures to a face.

11. A method of using the tube securement device of claim 1 comprising;
securing the first adhesive to a face;
securing the second adhesive to a tube.
